(19) United States
(12) Patent Application Publication
(10) Pub. No.: US 2025/0268563 A1
(43) Pub. Date: Aug. 28, 2025

(54) CARDIAC ULTRASONIC FINGERPRINTING: AN APPROACH FOR HIGH-THROUGHPUT MYOCARDIAL FEATURE PHENOTYPING

(71) Applicant: RUTGERS, THE STATE UNIVERSITY OF NEW JERSEY, New Brunswick, NJ (US)

(72) Inventors: Partho P. SENGUPTA, New Brunswick, NJ (US); Naveena YANAMALA, New Brunswick, NJ (US)

(21) Appl. No.: 19/207,238

(22) Filed: May 13, 2025

Related U.S. Application Data
(63) Continuation-in-part of application No. 17/617,465, filed on Dec. 8, 2021, now Pat. No. 12,315,621.
(60) Provisional application No. 62/864,771, filed on Jun. 21, 2019.

(51) Int. Cl. A61B 8/08; A61B 8/00

(52) U.S. Cl. CPC A61B 8/0883; A61B 8/48

(57) ABSTRACT

A system for identifying cardiac injury is provided herein. The system includes at least one computing device and at least one application executable on the at least one computing device. The application causes the computing device to extract a plurality of radiomic features from an ultrasound scan associated with a patient, determine one or more myocardial characteristics by applying the extracted plurality of radiomic features to one or more phenotyping models, and identify a cardiac injury associated with the patient based at least in part on the one or more myocardial characteristics and matching the extracted plurality of radiomic features to a patient cluster.

### CROSS-REFERENCE TO RELATED CASES

This application a continuation-in-part of U.S. application Ser. No. 17/617,465, which is a national stage entry pursuant to 35 U.S.C. § 371 of Patent Cooperation Treaty (PCT) international application No. PCT/US2020/037204, filed on Jun. 11, 2020, which claims priority to, and the benefit of, co-pending U.S. provisional application entitled “CARDIAC ULTRASONIC FINGERPRINTING: AN APPROACH FOR HIGH-THROUGHPUT MYOCARDIAL FEATURE PHENOTYPING” having Ser. No. 62/864,771, filed on Jun. 21, 2019, which are all hereby incorporated by reference in their entireties.

### BACKGROUND

Tissue characterization of myocardial pathology has been one of the greatest interests in the field of cardiac imaging. Advancement in noninvasive imaging techniques, especially cardiac magnetic resonance and echocardiography, has revealed that myocardial imaging features can be tightly associated with the pathological findings and provide valuable risk stratification. To that end, although cardiac ultrasound is considered the most accessible first-line imaging diagnostic tool that can accurately assess myocardial function and flow dynamics, ultrasound images endure significant impediments with regards to accurate tissue characterization. Although various attempts have been made to improve tissue characterization using B-mode image video-densitometry techniques and integrated backscatter, the limitations in tissue characterization using cardiac ultrasound have predominantly been due to variant intensities and image quality from echocardiography.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is an example of an ultrasound image and extracted basic statistic and spatial resampling variables from the ultrasound image in accordance with various embodiments of the present disclosure.

FIGS. 2A-2B are example of patient cluster models in accordance with various embodiments of the present disclosure.

FIG. 3 is an example of patient textures and texture-based phenotyping of left ventricular (LV) function in accordance with various embodiments of the present disclosure.

FIGS. 4A-4C are plots associated with automated supervised machine learning prediction for different prognosis in accordance with various embodiments of the present disclosure.

FIGS. 5A-5C are examples of identified myocardial fibrosis from textures and an example of classification of high-risk or low-risk myocardium clusters in accordance with various embodiments of the present disclosure.

FIG. 6 is an example of a graded patient cluster separated into high risk myocardium textures and low-risk myocardium textures in accordance with various embodiments of the present disclosure.

FIG. 7 is a schematic diagram illustrating associated with the machine learning pipeline for characterizing myocardial tissue based on extracted features from ultrasound images in accordance with various embodiments of the present disclosure.

FIG. 8 is a flowchart illustrating one example of functionality implemented as portions of the myocardial ultrasound fingerprinting application executed in a computing environment, in accordance to various embodiments of the present disclosure.

FIG. 9 is a schematic block diagram that provides one example illustration of a computing environment according to various embodiments of the present disclosure.

FIG. 10 is an example schematic associated with the process of myocardial fingerprinting according to various embodiments of the present disclosure.

FIG. 11 is an example drawing illustrated patient similarity based on myocardial texture features according to various embodiments of the present disclosure.

FIGS. 12A-12B are example graphical representations illustrating clinical outcomes between clusters according to various embodiments of the present disclosure.

FIGS. 13A-13C are example graphical representations illustrating the direct prediction of impaired cardiac function according to various embodiments of the present disclosure.

FIG. 14 is a block diagram illustrating the components of an ultrasound radiomics system for myocardial feature extraction and analysis in accordance with various embodiments of the present disclosure.

FIG. 15 is a flowchart illustrating a process data selection in accordance with an experiment demonstrating aspects of various embodiments of the present disclosure.

FIGS. 16A-16H are receiver operating characteristic (ROC) curves showing model performance metrics for myocardial infarction detection in accordance with various embodiments of the present disclosure.

FIG. 17 is a bar graph comparing performance metrics of different analysis methods for myocardial infarction detection in accordance with various embodiments of the present disclosure.

FIG. 18 is a heatmap visualization showing clustering patterns of radiomic features extracted from cardiac ultrasound images in accordance with various embodiments of the present disclosure.

FIG. 19 is a Manhattan plot showing the distribution of adjusted p-values for ultrasound radiomics features across different myocardial segments in accordance with various embodiments of the present disclosure.

FIGS. 20A-20F are cardiac imaging views showing parametric visualization of infarcted myocardium using different imaging techniques in accordance with various embodiments of the present disclosure.

FIGS. 21A-21B are density plots comparing ultrasonic feature distributions before and after harmonization across different ultrasound equipment vendors in accordance with various embodiments of the present disclosure.

FIG. 22A-22B are scatter plots comparing principal component analysis values of ultrasonic features across different ultrasound equipment vendors before and after harmonization in accordance with various embodiments of the present disclosure.

FIG. 23A-23B are graphs illustrating the effects of noise levels and gain adjustments on ultrasonic features extracted from different myocardial segments in accordance with various embodiments of the present disclosure.

FIG. 24A-24B are plots showing the distribution of gray level non-uniformity values across multiple myocardial segments under various noise conditions in accordance with various embodiments of the present disclosure.

FIG. 25A-25C are scatter plots demonstrating the correlation between cardiac magnetic resonance-derived infarct size measurements and ultrasound-based infarct size predictions in accordance with various embodiments of the present disclosure.

FIG. 26 presents Table 10, which includes data associated with the performance of machine learning models implemented in accordance with various embodiments of the present disclosure.

### SUMMARY

Aspects of the present disclosure are related to a myocardial imaging technique called myocardial ultrasonic fingerprinting that utilizes a radiomics-based approach and high-throughput computing on static cardiac ultrasound images.

In one aspect, among others, a system comprises at least one computing device and at least one application executable on the at least one computing device. When executed, the at least one application causes the at least one computing device to at least extract a plurality of radiomic features from an ultrasound image associated with a patient, determine one or more myocardial characteristics by applying the extracted plurality of radiomic features to one or more phenotyping models, and interpret a clinical significance associated with the patient based at least in part on the one or more myocardial characteristics and the extracted plurality of radiomic features.

In various aspects, among others, the ultrasound image comprises a plurality of ultrasound images. In various aspects, among others, the ultrasound image is a static image. In various aspects, among others, when executed, the at least one application causes the at least one computing device to at least identify a selection of a region of interest in the ultrasound image and the plurality of radiomics features are extracted within the region of interest.

In various aspects, among others, the radiomic features are extracted from a pixel-based pattern in the ultrasound image. In various aspects, among others, when executed, the at least one application further causes the at least one computing device to at least identify one or more myocardial textures based at in part on a clustering of the extracted plurality of radiomic features. In various aspects, among others, the ultrasound image is of a region of a heart. In various aspects, among others, the clinical significance is further based at least in part on matching the plurality of radiomic features to a patient cluster.

In various aspects, among others, the clinical significance is further based at least in part on matching the radiomic features to a gradient of a patient cluster. In various aspects, among others, the clinical significance comprises at least one of a ventricular malformation, a risk of advanced heart failure, myocardial fibrosis, one or more cardiac malignancies, or heart valve deterioration. In various aspects, among others, the one or more phenotyping models comprise at least one of a neural network classifier, a support vector machine (SVM) classifier, or a deep learning classifier. In various aspects, among others, when executed, the at least one application further causes the at least one computing device to at least select a portion of the plurality of radiomics features, select at least one of the one or more phenotyping models based at least in part on the portion of the plurality of radiomics features, and determine the one or more myocardial characteristics is based at least in part on the portion of the plurality of radiomics features and the at least one of the one or more phenotyping models.

In one aspect, among others, a method, comprises extracting, via at least one computing device, a plurality of radiomic features from an ultrasound image associated with a person, identifying, via the at least one computing device, one or more myocardial textures by applying the extracted plurality of radiomic features to at least one phenotyping model, and determining, via the at least one computing device, at least one condition associated with the person based at least in part on the one or more myocardial textures and the extracted plurality of radiomic features.

In various aspects, among others the method further comprising: comparing, via the at least one computing device, the one or more myocardial textures to at least one phenotype cluster for at least one known condition; and determining the at least one condition is based at least in part on the one or more myocardial textures being matched with one or more of the at least one phenotype cluster.

In various aspects, among others, the method further comprises obtaining, via at least one computing device, the ultrasound image from an ultrasound capturing device in data communication with the at least one computing device. In various aspect, among others, extracting the plurality of radiomic features from the ultrasound image further comprises detecting pixel-based patterns in the ultrasound image. In various aspect, among others, the method further comprises identifying, via the at least one computing device, at least one selected region of interest in the ultrasound image, wherein the plurality of radiomic features are extracted from the at least one selected region of interest in the ultrasound image.

In various aspects, among others, the at least one condition comprises at least one of a ventricular malformation, a risk of advanced heart failure, myocardial fibrosis, one or more cardiac malignancies, or heart valve deterioration. In various aspect, among others, the one or more phenotyping models comprise at least one of a neural network classifier, a support vector machine (SVM) classifier, or a deep learning classifier. In various aspect, among others, the ultrasound image comprises a static two-dimensional cardiac ultrasound image.

In one aspect, among others, a system comprises at least one computing device and at least one application executable on the at least one computing device. When executed, the at least one application causes the at least one computing device to at least extract a plurality of radiomic features from an ultrasound scan associated with a patient, determine one or more myocardial characteristics by applying the extracted plurality of radiomic features to one or more phenotyping models, and identify a cardiac injury associated with the patient based at least in part on the one or more myocardial characteristics and matching the extracted plurality of radiomic features to a patient cluster.

In various aspects, among others, the cardiac injury is a myocardial infarction, and the system quantifies a size of an infarct associated with the myocardial infarction. In various aspects, among others, the system locates infarcted myocardium based at least in part on the one or more myocardial characteristics and creates a parametric map of the infarcted myocardium. In various aspects, among others, the system extracts the plurality of radiomic features from a plurality of ultrasound scans associated with the patient, the plurality of ultrasound scans comprising a plurality of views.

In various aspects, among others, the plurality of radiomic features comprise dynamic features and static features. In various aspects, among others, the system identifies one or more myocardial textures based at least in part on a clustering of the extracted plurality of radiomic features. In various aspects, among others, the identification of the myocardial infarction is further based at least in part on matching the radiomic features to a gradient of the patient cluster.

In various aspects, among others, the system determines a global-to-local association and estimates local cardiac damage based at least in part on the global-to-local association. In various aspects, among others, the system estimates global cardiac damage based at least in part on the extracted plurality of radiomic features.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### DETAILED DESCRIPTION

Disclosed herein are various embodiments related to characterizing pathological myocardial tissue using computational processes to acquire, process, and visualize data. In particular, systems and methods of the present disclosure relate to a myocardial imaging technique called myocardial ultrasonic fingerprinting that utilizes a radiomics-based approach and high-throughput computing on static cardiac ultrasound images. The technique allows for the extraction of pixel-based information from noisy multidimensional static images and isolates quantitative features of myocardial tissue. In addition, machine learning techniques accompany the data analysis to enhance the signal-to-noise ratio from complex multidimensional data. In this manner, the fingerprinting technique of the present disclosure elucidates numerous features that can serve as predictors of cardiovascular pathology as well as prognostic indicators measuring treatment response.

Cardiovascular disease accounts for one in every four deaths in the United States-approximately 610,000 people every year, according to the Centers for Disease Control. Tissue characterization of myocardial pathology has been an area of intense research and development due to the rising incidence of cardiovascular conditions and the growing geriatric population. Many noninvasive imaging techniques are used to associate myocardial imaging features with pathological assessments. While cardiac ultrasound is considered the first-line noninvasive diagnostic imaging tool to assess myocardial function, ultrasound images do not always display accurate tissue characterization. This has mainly been due to variant intensities that affect the signal-to-noise ratio, which in turn affects the quality of images from echocardiography.

Cardiac ultrasound imaging is considered the most accessible and first-line imaging tool with accurate assessment of myocardial function and flow dynamics. Furthermore, radiomics is a method that extracts large number of features from radiographic medical images using data-characterization algorithms. These features, termed radiomic features, have the potential to uncover disease characteristics that fail to be appreciated by the naked eye. Groups have utilized the radiomics approach to analyze ultrasound images of breast tissue and found the radiomics approach could differentiate between different types of cancers. Moreover, other groups have analyzed Computed Tomography (CT) and myocardial perfusion Singly Photon Emission Control Tomography (SPECT) images for cardiac purposes using a radiomics-based approach. Typical myocardial texture is visually distinguishable with ultrasound images. However, image quality and texture of ultrasound images may vary significantly. The variance of image quality and texture of ultrasound images is induced by different factors including, for example, patient factors, existence of a good acoustic window, machine settings, and skill of sonographers, thereby preventing reproducible quantitative myocardial texture analysis.

According to various embodiments, myocardial ultrasonic fingerprinting characterizes pathological myocardial tissue using cutting-edge computational processes to acquire, process, and visualize data. Myocardial ultrasonic fingerprinting uses multiple material properties and a radiomics-based approach to parameter mining that identifies pathological changes earlier than traditional qualitative imaging. The radiomics-based approach improves the predictive accuracy of the diagnosis, and the machine learning techniques that accompany the data analysis provide a method to enhance the signal-to-noise ratio from complex, multidimensional data. The pathological features that the present disclosure could detect include left ventricular (LV) malformations, risk of advanced heart failure, and myocardial fibrosis. The technology could also potentially detect cardiac malignancies and heart valve deterioration.

According to various embodiments of the present disclosure, radiomic-based myocardial ultrasonic fingerprinting improves traditional tissue imaging techniques using echocardiography and provides a reliable prediction of cardiological issues. The disclosed method is more precise than other tissue imaging techniques because radiomic-based myocardial ultrasonic fingerprinting allows for extraction of information from noisy multidimensional medical images and identifies quantitative features of myocardial tissue from static cardiac ultrasound images.

According to various embodiments of the present disclosure, the radiomic-based myocardial ultrasonic fingerprinting further improves traditional myocardial tissue characterizations by extracting maximal information from standard-of-care images using high-throughput computing. Compared with other conventional approaches, radiomic-based fingerprinting can extract at least sixty-four (64) distinct variables from any predefined region of a cardiac ultrasonic image, or at least 256 distinct variables from a single patient. Subsequently, in view of traditional methods of characterizations requiring sonographers, texture-based machine learning phenotyping is applied to the extracted variables to identify patient clusters and thereby diagnose various pathological conditions including, but not limited to, left ventricular (LV) malformations, risk of advanced heart failure, myocardial fibrosis, cancer taxonomy, predictive therapeutic response, gene expression, and/or other conditions. Furthermore, according to various embodiments, the present disclosure allows for earlier identification of pathological cardiovascular changes than compared to traditional qualitative imaging techniques, thereby improving diagnostic, prognostic, and predictive accuracy.

According to various embodiments of the present disclosure, radiomics-based myocardial ultrasonic fingerprinting further improves on traditional approaches of myocardial tissue characterization by identifying myocardial fibrosis using radiomic-based clustering. Although this feature may be extracted from static images, similar accuracy is achieved as full frame derived global longitudinal strain (GLS) values for identifying myocardial fibrosis. Thus, radiomic-based fingerprinting can extract important image features. Therefore, excellent diagnostic accuracy can be achieved from the combination of GLS and radiomic features.

According to various embodiments, radiomic-based myocardial ultrasonic fingerprinting improves traditional approaches of myocardial tissue characterization by being able to reliably predict cardiological issues without requiring the storage, processing, and use of dense movie files for analysis. The radiomics-based fingerprinting approach may be applied to static cardiac ultrasound images. As a result, data storage usage is greatly reduced, thereby significantly reducing costs to users.

Reference will now be made in detail to the description of the embodiments as illustrated in the drawings, wherein like reference numbers indicate like parts throughout the several views.

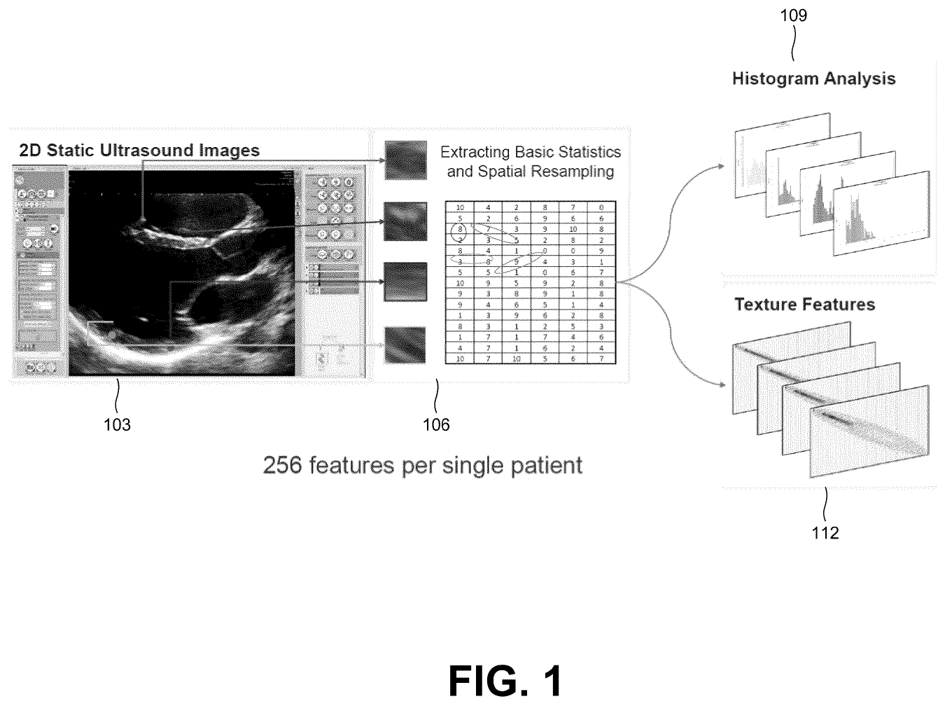

Turning to FIG. 1, shown is an example of a two-dimensional (2D) ultrasound image 103 and the information that may be extracted from the 2D ultrasound image 103, according to various embodiments of the present disclosure. From the 2D ultrasound image 103, quantitative radiomic texture indices are extracted in the form of basic statistics and spatial resampling variables 106. In this example, multiple (e.g., 256) radiomic texture indices may be extracted from an image for a single patient. From the extracted basic statistics and spatial resampling variables 106, histogram analysis 109 may be performed and texture features 112 may be characterized. Those texture features can include Gray Level Co-occurrence Matrix (GLCM), Gray Level Zone Length Matrix (GLZLM), Gray Level Run Length Matrix (GLRLM), Neighborhood Grey Level Different Matrix (NGDLDM), and/or other type of texture feature as can be appreciated. Each feature (e.g., matrix) carries unique information. For example, GLCM takes into account the arrangements of pairs of voxels to calculate textural indices. GLZLM provides information on the size of homogeneous zones for each grey-level. GLRLM gives the size of homogeneous runs for each grey level. NGLDM corresponds to the difference of grey-level between one voxel and its neighbors.

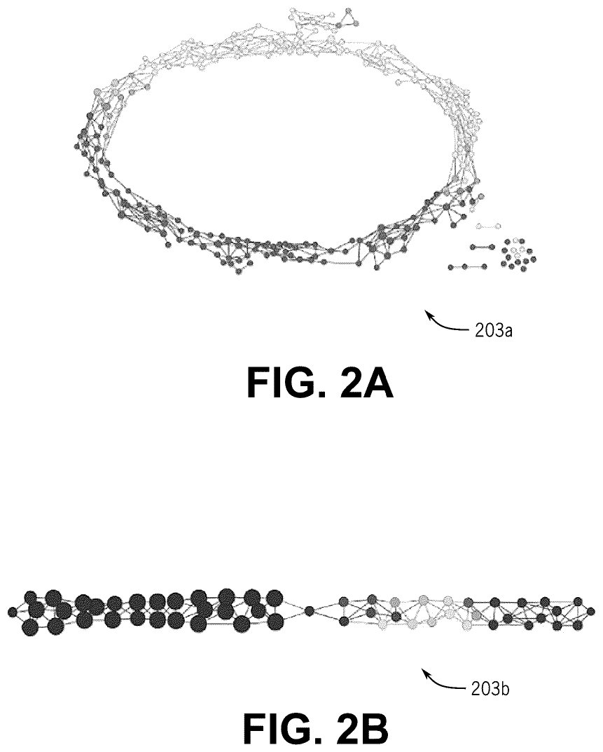

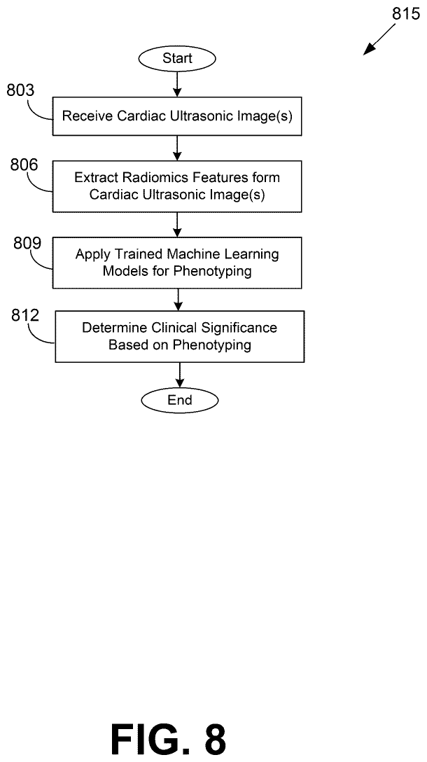

In FIGS. 2A and 2B, shown are examples of patient cluster mappings 203 (e.g., 203a, 203b) for a cardiac issue, according to various embodiments of the present disclosure. Once basic statistics and spatial resampling variables 106 (FIG. 1) are extracted from a 2D ultrasound image 103 (FIG. 1), the myocardial ultrasonic fingerprinting application 815 (FIG. 8) matches the extracted variables to a patient cluster mapping 203. The patient cluster mappings 203 are identified using unsupervised machine learning techniques where similar patients are aggregated in nodes that are close to each other. In particular, the patient cluster mappings 203 can be identified using a patient similarly analysis such as, for example, a topological data analysis, as shown in FIGS. 2A and 2B. The geometry identifies groups of similar patients and their significance is understood by colorizing clinical features and outcome that have not been part of the initial model building.

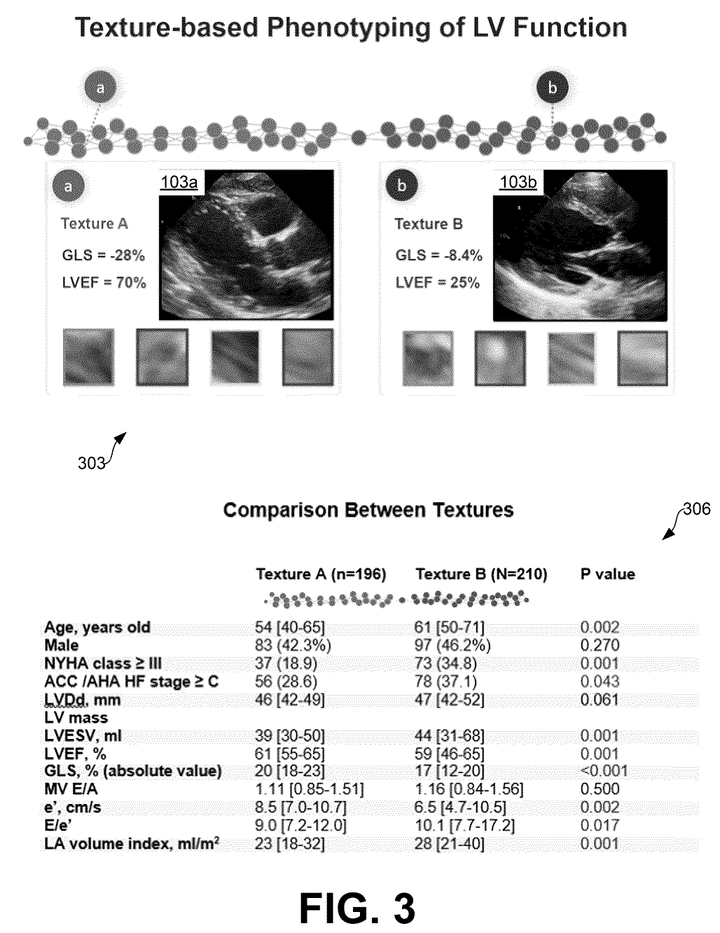

In FIG. 3, shown is an example of texture-based phenotyping 300 for left ventricle function and a table 306 showing a comparison between texture A and B, according to various embodiments of the present disclosure. The texture-based phenotyping for the left ventricle function can identify different texture features, such as GLS and Left Ventricular Ejection Fraction (LVEF), from pixel-based patterns in a 2D cardiac ultrasonic image 103 (e.g., 103a, 103b), thereby identifying quantitative features of myocardial tissue with more predictive accuracy than conventional methods, and allowing for identification of pathological cardiovascular changes earlier than traditional qualitative imaging. The texture-based phenotyping of LV function as shown in FIG. 3 may also be used to perform a comparison between textures 306 to aid in identifying various cardiac conditions.

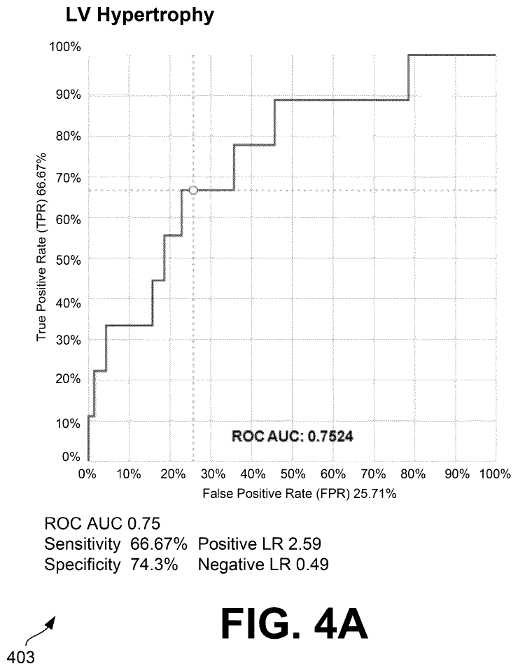

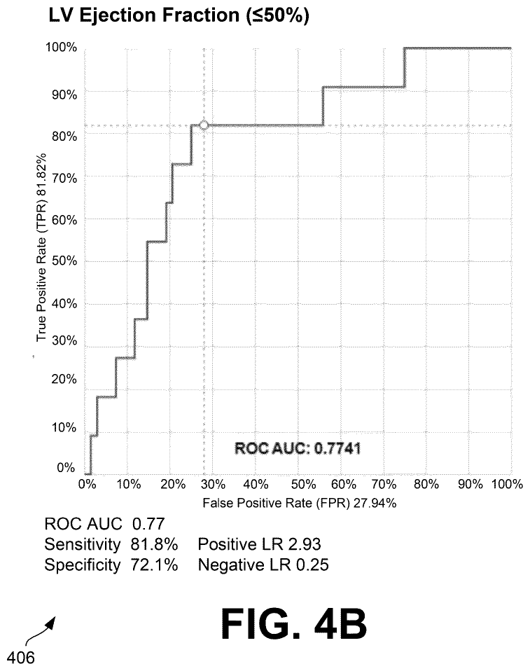

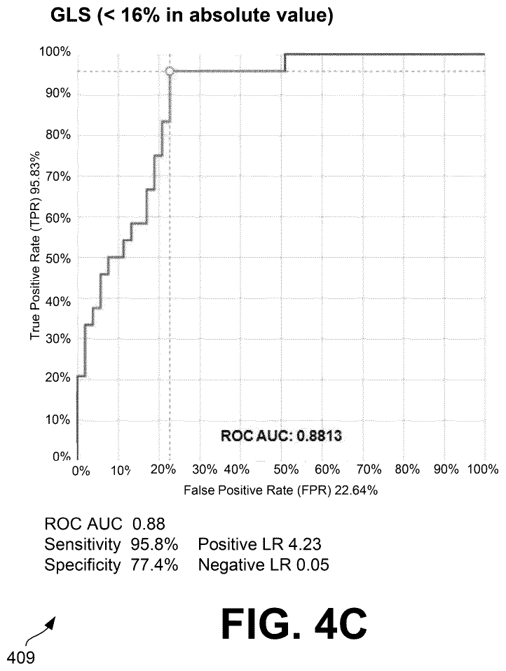

Turning to FIGS. 4A-4B, shown are example plots illustrating automated supervised machine learning prediction measurement for three representative cardiac features. According to various embodiments, FIG. 4A illustrates a plot 403 associated with LV hypertrophy, FIG. 4B illustrates a plot 406 associated with LVEF 406, and FIG. 4C illustrates a plot 409 associated with GLS. The automated supervised machine learning prediction of the present disclosure, and as shown in FIGS. 4A-4C, provides an evaluation metric for various cardiac features. In addition. the automated supervised machine learning prediction allows for monitoring of sensitivity, specificity, and likelihood ratio (LR) for various cardiac texture.

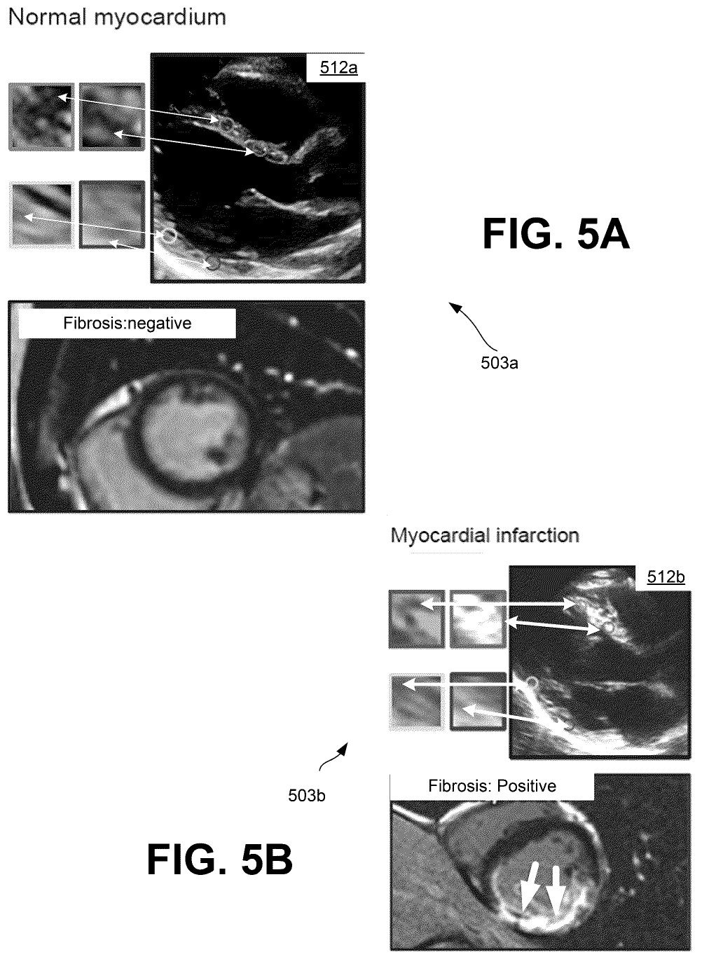

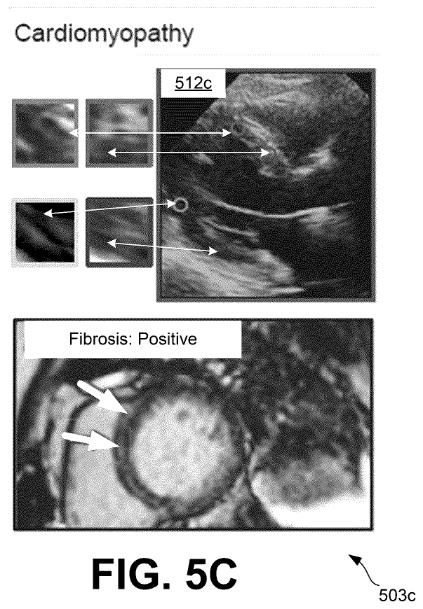

In FIGS. 5A-5C shown are examples of myocardial fibrosis textures 503 (e.g., 503a, 503b, 503c) for different patients, according to various embodiments of the present disclosure. In particular, FIGS. 5A-5C illustrate static cardiac ultrasound images 512 (e.g., 512a, 512b, 512c) of different patients and illustrate different conditions corresponding to normal myocardium, myocardial infarction, and cardiomyopathy. According to various embodiment, the myocardial ultrasonic fingerprinting application 815 (FIG. 8) can be trained to identify negative or positive myocardial fibrosis textures through radiomic-based clustering of a static cardiac ultrasound image 512. Although these features are extracted from a static cardiac ultrasound image 512, similar accuracy as full-frame GLS values for identifying myocardial fibrosis is achieved. Thus, exemplifying excellent diagnostic accuracy from the combination of GLS and radiomic features. Therefore, radiomic-based fingerprinting can extract important hidden image features that are tightly associated with cardiac disease conditions.

### Example 1

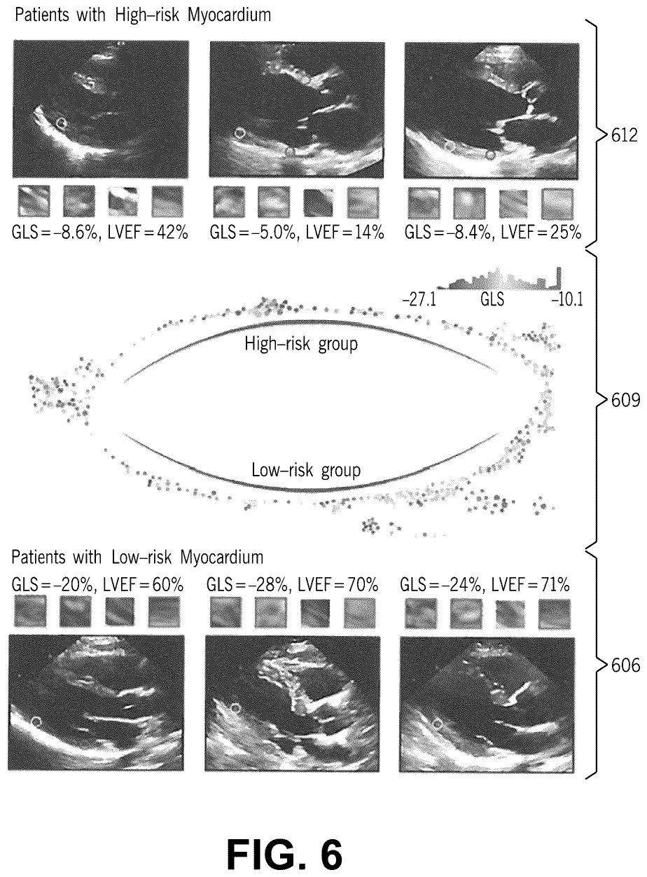

Preliminary evidence and proof-of-concept was demonstrated in a recent investigation involving two hundred fifty-six (256) radiomic texture indices from images taken from 446 patients. The radiomic data was compared with conventional echocardiography and 2D speckle tracking derived global longitudinal strain. In a subgroup of forty patients undergoing cardiac magnetic resonance (CMR), high-risk fingerprint was subsequently assessed in total 160 left ventricular (LV) segments for predicting the presence of myocardial fibrosis as defined by late gadolinium-enhanced CMR. As shown in FIG. 6, topological data analysis clustered the patients with high and low-risk myocardial fingerprint in an unsupervised manner.

The high-risk pathological features were associated with conventional markers of LV remodeling including LV end-diastolic and systolic volumes, ejection fraction, and impaired global longitudinal strain. Furthermore, the high-risk fingerprint predicted presence of advanced heart failure (ACC/AHA stage≥C) and symptoms (NYHA class≥III). In patients undergoing CMR, the high-risk fingerprint was an independent predictor of fibrosis, and adding fingerprint information to global longitudinal strain improved prediction of myocardial fibrosis. Taken together, these results indicated that radiomic-based cardiac ultrasound fingerprinting identifies high-risk features associated with LV remodeling in early and advanced clinical stages of heart failure.

FIG. 6 illustrates an example of classification of various cardiac textures based on the above experiment according with high-risk myocardium 603 and low-risk myocardium 606 within a patient cluster 609 based on phenotyping, according to various embodiments of the present disclosure. Although the classification of FIG. 6 identifies two clusters (e.g., high risk, low risk), extracted radiomic-based fingerprinting may be classified with any number of graded patient clusters to identify a variety of clinically significant indications.

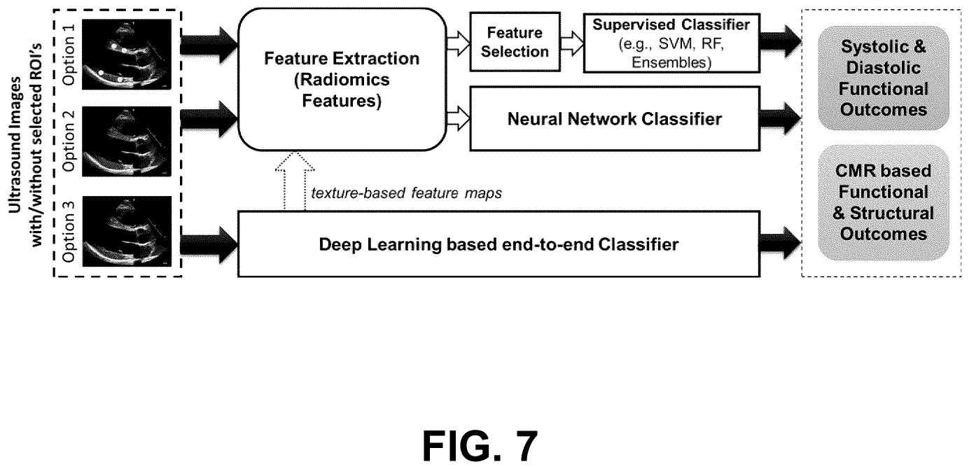

Turning now to FIG. 7, shown is a sample schematic associated with the machine learning pipeline for cardiac ultrasonic fingerprinting, according to various embodiments of the present disclosure. In particular, FIG. 7 relates to various techniques that can be used to generate myocardial ultrasonic fingerprints that can be used to identify cardiac characteristics that can be used to diagnosis and treat patients.

In various embodiments, features associated with the obtained images can be extracted from static cardiac ultrasound images 103, 512. In some embodiments, the features can be extracted based on selected regions of interest within the image. In other embodiments, the features can be extracted without a selection of a region of interest. The features can correspond to quantitative radiomic texture indices are extracted in the form of basic statistics and spatial resampling variables.

The features can be applied to trained machine learning models in the form of deep learning-based classifiers, supervised classifiers, and neural network classifiers for high-throughput myocardial feature phenotyping. Accordingly, the trained models of the present disclosure can map the extracted features to various myocardial feature phenotypes. In particular, the analysis of the extracted features from the images and application of the extracted features with respect to the trained models can be used to identify various cardiac characteristics including, for example, systolic functional characteristics, diastolic functional characteristics, cardiac magnetic resonance (CMR) based functional characteristics, CMR based structural characteristics, and/or other cardiac characteristics

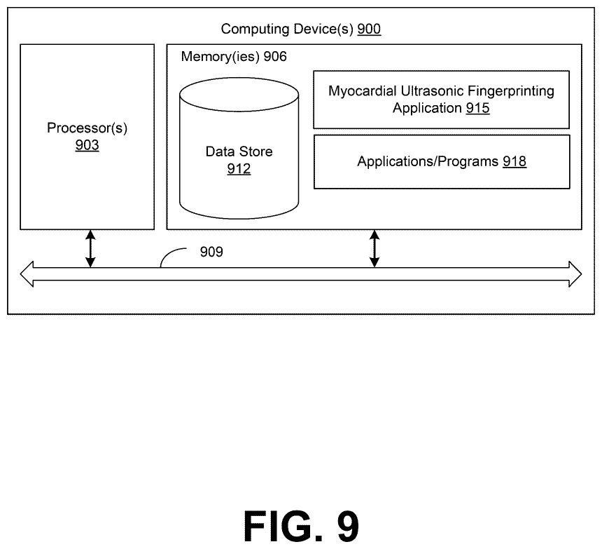

Turning now to FIG. 8, shown is an example of a flowchart illustrating one example of the operation of a portion of the myocardial ultrasonic fingerprinting application 915 (FIG. 9) executed in a computing environment according to various embodiments of the present disclosure. It is understood that the flowchart of FIG. 8 provides merely an example of the many different types of functional arrangements that may be employed to implement the operation of the portion of the myocardial ultrasonic fingerprinting application 915 (FIG. 9) as described herein. As an alternative, the flowchart of FIG. 8 may be viewed as depicting an example of elements of a method implemented in the computing environment according to one or more embodiments.

Beginning with box 803, the myocardial ultrasonic fingerprinting application 815 (FIG. 8) obtains one or more cardiac ultrasound images 103, 512 of a patient. For example, the myocardial ultrasonic fingerprinting application 815 could receive a single 2D cardiac ultrasound image 103 (FIG. 1) of a region of the heart. As another example, the myocardial ultrasonic fingerprinting application 815 could receive multiple images 103, 512 of one or more regions of the heart. Upon receiving the images 103, 512, the myocardial ultrasonic fingerprinting application 815 may extract and temporarily store all of the images for further processing.

Moving on to box 806, the myocardial ultrasonic fingerprinting application 815 extracts radiomics features from cardiac ultrasonic images 103, 512. For example, the myocardial ultrasonic fingerprinting application 815 can identify pixel-based patterns from a 2D cardiac ultrasound image that cannot be appreciated by the human eye. The myocardial ultrasonic fingerprinting application 815 can extract radiomic features from the 2D cardiac ultrasound image 103, 512 in the form of basic statistics and spatial resampling variables 106 (FIG. 1). As another example, the myocardial ultrasonic fingerprinting application 815 (FIG. 8) can extract radiomic features 106, 109, 112 (FIG. 1) from a plurality of 2D cardiac ultrasound images 103 (FIG. 1) and use the extracted radiomic features to perform histogram analysis 109 and characterize certain myocardial texture features 112 (FIG. 1) despite variations in quality and texture of 2D cardiac ultrasound images 103 (FIG. 1). In some embodiments, the extracted features may correspond to selected regions of interest. For example, a user may select various regions of interest in a given image that are to be analyzed. In other embodiments, there are no selected regions of interest and the image is analyzed as a whole without a selection of one or more regions of interest.

Proceeding to box 809, the myocardial ultrasonic fingerprinting application 815 (FIG. 8) can apply the extracted radiomic features 106, 109, 112 (FIG. 1) to trained machine learning models for myocardial phenotyping. In some embodiments, the myocardial ultrasonic fingerprinting application 815 can apply the extracted radiomic features 106, 109, 112 (FIG. 1) to selected machine learning phenotyping models and match the phenotype to a patient cluster 203 (FIG. 2) for an identified cardiac issue. As another example, myocardial ultrasonic fingerprinting application 815 (FIG. 8) can apply the extracted radiomic features 106, 109, 112 (FIG. 1) to selected machine learning phenotyping models and match the phenotype to at least one of a plurality of grades of patient clusters 609 (FIG. 6) for multiple cardiac issues or for different grades of a particular cardiac issue.

Moving on to box 812, the myocardial ultrasonic fingerprinting application 815 can identify phenotypic features based at least in part on extracted radiomic features 106, 109, 112 (FIG. 1) of a 2D cardiac ultrasound image 103 and interpret the clinical significance (e.g., left ventricular (LV) malformations, risk of advanced heart failure, myocardial fibrosis, cardiac malignancies, heart valve deterioration, etc.). In another example, the myocardial ultrasonic fingerprinting application 815 (FIG. 8) can interpret clinical significance based at least in part on the 2D cardiac ultrasound image's 103 match to a patient cluster 203 or at least one of plurality of different grades of patient clusters 609. As another example, the extracted phenotypic features from the 2D cardiac ultrasound images can be used in bioinformatics platform analysis model to identify the group the patient belongs to.

Thereafter, the process proceeds to completion.

With reference now to FIG. 9, shown is one example of at least one computing device 900 (e.g., an interfacing device, central server, server, or other network device) that performs various functions of the myocardial ultrasonic fingerprinting algorithms in accordance with various embodiments of the present disclosure. Each computing device 900 includes at least one processor circuit, for example, having a processor 903 and a memory 906, both of which are coupled to a local interface 809. To this end, each computing device 900 may be implemented using one or more circuits, one or more microprocessors, microcontrollers, application specific integrated circuits, dedicated hardware, digital signal processors, microcomputers, central processing units, field programmable gate arrays, programmable logic devices, state machines, or any combination thereof. The local interface 909 may comprise, for example, a data bus with an accompanying address/control bus or other bus structure as can be appreciated. Each computing device 900 can include a display for rendering of generated graphics such as, e.g., a user interface and an input interface such, e.g., a keypad or touch screen to allow for user input. In addition, each computing device 800 can include communication interfaces (not shown) that allows each computing device 900 to communicatively couple with other communication devices. The communication interfaces may include one or more wireless connection(s) such as, e.g., Bluetooth or other radio frequency (RF) connection and/or one or more wired connection(s).

Stored in the memory 906 are both data and several components that are executable by the processor 903. In particular, stored in the memory 906 and executable by the processor 903 is the myocardial ultrasonic fingerprinting application 915, and/or other applications 918. Also stored in the memory 903 may be a data store 912 and other data. It is understood that there may be other applications that are stored in the memory 906 and are executable by the processor 903 as can be appreciated. Where any component discussed herein is implemented in the form of software, any one of a number of programming languages may be employed such as, for example, C, C++, C#, Objective C, Java©, JavaScript®, Perl, PHP, Visual Basic©, Python®, Ruby, Delphi®, Flash®, LabVIEW® or other programming languages.

A number of software components are stored in the memory 906 and are executable by the processor 903. In this respect, the term “executable” means a program file that is in a form that can ultimately be run by the processor 903. Examples of executable programs may be, for example, a compiled program that can be translated into machine code in a format that can be loaded into a random access portion of the memory 906 and run by the processor 903, source code that may be expressed in proper format such as object code that is capable of being loaded into a random access portion of the memory 906 and executed by the processor 903, or source code that may be interpreted by another executable program to generate instructions in a random access portion of the memory 906 to be executed by the processor 903, etc. An executable program may be stored in any portion or component of the memory 906 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 906 is defined herein as including both volatile and nonvolatile memory and data storage components. Volatile components are those that do not retain data values upon loss of power. Nonvolatile components are those that retain data upon a loss of power. Thus, the memory 806 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 903 may represent multiple processors 903 and the memory 906 may represent multiple memories 906 that operate in parallel processing circuits, respectively. In such a case, the local interface 909 may be an appropriate network that facilitates communication between any two of the multiple processors 903, between any processor 903 and any of the memories 906, or between any two of the memories 906, etc. The local interface 909 may comprise additional systems designed to coordinate this communication, including, for example, performing load balancing. The processor 903 may be of electrical or of some other available construction.

Although the myocardial ultrasonic fingerprinting application 915, other application(s) 918, and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits having appropriate logic gates, or other components, etc. Such technologies are generally well known by those skilled in the art and, consequently, are not described in detail herein.

Also, any logic or application described herein, including the myocardial ultrasonic fingerprinting application 915 and/or application(s) 918, that comprises software or code can be embodied in any non-transitory computer-readable medium for use by or in connection with an instruction execution system such as, for example, a processor 803 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system. The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

### Example 2

A rise in cardiovascular risk factors, improved survival rate from ischemic heart disease, and population-ageing have contributed to the increasing global burden of heart failure. An important step to prevent the progression of heart failure includes early detection of left ventricular (LV) remodeling—a process driven by architectural cellular and interstitial changes in the myocardium and identified clinically as global changes in LV size, geometry, and function. Studies have shown that the degree of LV remodeling has a strong correlation with the impact of particular drugs or device therapies as well as with clinical outcomes.

Recent advancements in cardiac magnetic resonance (CMR) have revealed that myocardial tissue imaging characteristics alter under various cardiac conditions which reflect structural LV remodeling, including fibrosis, increased extracellular volume, and altered fibre orientation. Cardiac ultrasound is not currently utilized clinically for myocardial tissue characterization although previous studies have reported that the intensity of the ultrasound backscatter is related to the physical properties of the myocardium and is influenced by tissue components (e.g., collagen, water, fat). Moreover, there has been limited information regarding the specific application of texture-based analysis for cardiac ultrasound imaging.

The recent developments in image analysis and novel bioinformatics approaches have augmented methods that can extract information from the texture in a still image. The application of such texture-based image analysis has been increasingly utilized as a key function in various image processing applications such as automated inspection, document processing, radiology image processing, and content-based image retrieval. Such techniques may also have direct relevance for cardiac ultrasound techniques like speckle tracking echocardiography where myocardial motion is analyzed using frame-by-frame tracking of natural acoustic markers (often referred in literature as “speckles”, “patterns”, or “fingerprints”). A functional unit (kernel) of speckles generated from ultrasound-tissue interactions (e.g., reflections, interference, and scattering) is unique, allowing software to track itself during the entire cardiac cycle. Thus, an ultrasound texture of myocardium may carry unique and specific information of the indexed myocardium.

According to various embodiments, the present disclosure presents the development and validation of a novel approach that combines the texture-based informatics of myocardium with machine learning techniques. First, texture-based tissue features are extracted from still ultrasound images and the association of texture feature-based patient phenotypes with LV remodeling are identified. Subsequently, the value of texture-based supervised machine learning models in predicting LV systolic dysfunction and the presence of myocardial fibrosis in a remodeled LV is illustrated.

### Materials and Methods

### Study Participants

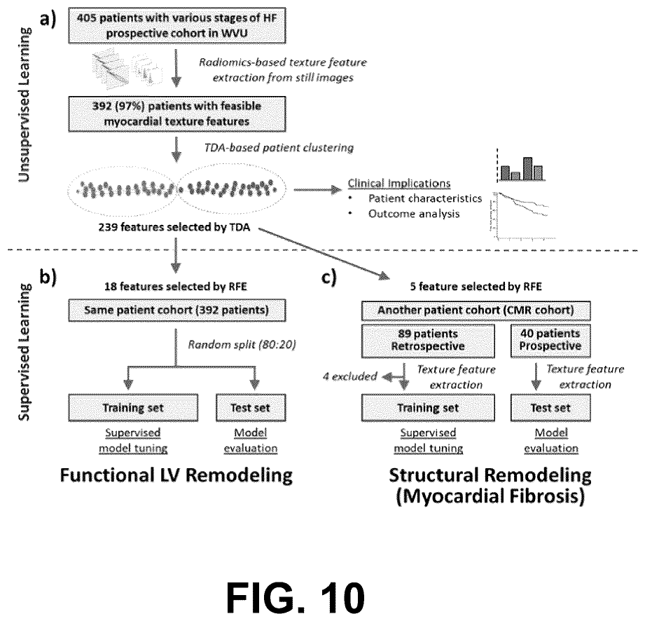

This study consisted of three parts. The detailed study design is presented in FIG. 10.

### Unsupervised Phenotyping Based on Texture Features (FIG. 10 (a)).

405 patients were pooled from three prospective studies conducted at West Virginia University between August 2017 and September 2018. Those studies used echocardiography as a reference standard of LV function and were evaluating the value of a surface ECG algorithm to predict diastolic dysfunction (n=196). This study included adult (>18 years old) subjects who underwent ECG and echocardiography on the same day; a probe for estimating pulmonary artery pressure from chest wall (n=145). This study included adults older than 18 years old, admitted to the hospital for HF who had an echocardiogram performed within 48 hours of presentation, and a software for the assessment of intracardiac flow (n=64), which included consecutive adult patients referred for LV function assessment. The common exclusion criteria for all the three studies included patients with inadequate echocardiographic views and patients with chest deformities. Myocardial texture feature extraction was feasible in 392 patients. An unsupervised machine learning using topological data analysis was used for aggregating patients with similar textural properties and compared the patient characteristics, cardiac function, and outcome between the phenogroups.

### Supervised Learning-Based Prediction of LV Remodeling (FIG. 10 (b))

The 392 patient cohort as described above was used to develop supervised machine learning models for predicting functional markers of LV remodeling (impairment in LV ejection fraction [LVEF] and global longitudinal strain [GLS]), the cohort was randomly divided into a training (80%) and test (20%) set. Then, machine-learning models were trained in the training set (with cross-validation) and subsequently evaluated in the test set.

### Supervised Learning-Based Prediction of Myocardial Fibrosis (FIG. 10 (c))

To assess the value of texture features for predicting the presence of CMR delineated myocardial fibrosis, 89 patients who underwent clinically indicated CMR and cardiac ultrasound within 48 hours between July 2017 and December 2018 were retrospectively identified. Exclusion criteria were patients with inadequate echocardiographic views, patients with chest deformities, and patients who underwent CMR without gadolinium contrast. The retrospective cohort was used to train machine learning models with cross-validation and the developed model was tested in 40 prospective patients who were enrolled with the same inclusion/exclusion criteria.

### Data Collection

The New York Heart Association (NYHA) functional class and the heart failure stages defined by the American College of Cardiology and the American Heart Association were used to investigate clinical severity. Major adverse cardiac event (MACE) was predefined as the composite of cardiac death, hospitalization due to myocardial infarction, acute coronary syndrome, heart failure, and arrhythmias and were tracked on an electronic chart and/or telephone interview. The Meta-Analysis Global Group in Chronic (MAGGIC) heart failure risk score was calculated as previously reported. All enrolled patients underwent comprehensive 2-dimensional echocardiography using commercially available ultrasound equipment (Vivid-9/95, GE Healthcare; iE-33, Philips Healthcare; and LISENDO 880, Hitachi Healthcare) with 1-5 MHz phased array probes. Ultrasound images were stored in a DICOM format on the institute's local Picture Archiving and Communication System (PACS). Conventional echocardiographic parameters were analyzed per under the current guidelines. LVEF was measured using 2D disk methods at end-diastole and end-systole. Speckle tracking strain analysis was performed offline using vendor-free software (ImageArena, TomTec Inc.) by observers who were blinded to other information, including the texture-based tissue features. The longitudinal strain was calculated using apical 4-, 2-, and long-axis views, and the averaged value was reported as the GLS. CMR was performed using a 1.5 Tesla scanner (MAGNETOM Arena, Siemens Healthineers, Erlangen, Germany). Late gadolinium enhancement imaging was performed in all subjects in accordance with standard clinical protocols. Late gadolinium enhancement was defined by hyper-enhanced pixels with signal intensities of five standard deviations above the mean of normal myocardium. Patients were considered to have myocardial fibrosis in the studied segments if positive late gadolinium enhancement was seen in any of the anteroseptal and posterior wall myocardial segments (corresponding to the segments where ultrasound ROIs were placed for extracting texture features).

### Quantitative Texture-Based Tissue Feature Extraction

Texture-based tissue features of myocardium were extracted from still images of traditional parasternal long axis views using LIFEx software v4.5. This technique of texture-based feature extraction has been popularized in radiology and referred to as ‘radiomics’. Using two still frames, an end-diastolic and an end-systolic frame, circular regions of interest (ROIs) including 257 pixels (4-9 millimeter (mm) in diameter) per each, were placed at the basal and mid-segments of the interventricular septum and the left ventricular posterior wall, respectively. The basal and mid-segments were defined as the level of the mitral valve leaflet tips and the papillary muscle. The ROI contents were first resampled in 64 discrete values using the formula:

R(x)=round(64*[I(x)−min ROI intensity]/[max ROI intensity−min ROI intensity])

Where R(x) is the resampled value of pixel x, I(x) is the intensity of pixel x in the original image, and max and min intensity are the maximum and minimum intensities in the ROI, respectively. The software extracted forty-one texture features, or radiomics features, from each ROI, including first-order statistics such as the maximum, minimum, standard deviation, and the mean value of intensity and histogram features, and second-order indices such as the gray-level co-occurrence matrix (GLCM), gray-level run length matrix (GLRLM), neighboring gray-level dependence matrices (NGLDM), and gray-level zone length matrices (GLZLM).

### Feature Phenotyping Using Topological Data Analysis

A total of 328 texture features extracted during diastole and systole were included in the topological data analysis using Ayasdi Workbench v7.4 (Ayasdi Inc., Menlo Park, California). Topological data analysis is a novel mathematical and data analysis approach that establishes the topological and geometrical structure of the data to garner information and patterns from the features in a patient-patient similarity network.

In a topological data analysis-based patient similarity network, patients with similar features (in this study, texture-based tissue features) form a node or a dot, and adjacent nodes, including similar patients, are connected with edges or lines. Accordingly, the relative distance between nodes (more precisely, the minimum number of edges between nodes) represents the similarity of features between the nodes. Thus, clusters or groups of patients with similar features can be identified based on the shape of the network. This notion of linking the shape to meaning using tuning based on Bayesian parameter optimization using an optimization technique called sequential model-based algorithm configuration]. This process was performed with Monte-Carlo cross validation.

Finally, several models with the highest performance were selected and their prediction probabilities were averaged to create an ensemble model (fusion model), which were evaluated in the hold-out (not used in the training process) test set. Such techniques of making fusion models help combining diverse and independent models for reducing the generalization error.

### Topical Data Analysis—Technical Details

Topological data analysis (TDA) is a novel data visualization technique and a framework for machine learning that is based on the mathematical concept of topology—a subfield of geometry to study the shape and the topological space. It pertains to the analysis of the space that is invariant under certain transformations in a continuous map of f:X→Y from topological space X to topological space Y. Therefore, there are three fundamental invariants of topology that is pertinent in the properties of the topological space: coordinate invariance, deformative invariance, and compressed representation.

Coordinate invariance of the topological space only concerns the property of the shape rather than the coordination and the arrangement of the object. The orientation of the object possesses no value or information as the shape of the object is topologically same. Similarly, the discernment of the object is consistent in the topological space regardless of its stretching or compression to preserve its deformation invariance. Finally, the compressed representation of the topology concerns with connectivity and continuity of the object to provide the summary and succinct description of the shape.

In TDA, two types of parameters are required to generate the network. First, the finite dataset is used to construct the point cloud in the manifold using similarity measurements by applying one of various metrics such as Euclidean distance, binary Jaccard, Hamming distance, or correlation, to name a few. Second, the function that describes the distribution of the data to create a representative node based on the overlapping bins of the dataset. These vital parameters are referred to as metric and lens, respectively. Unlike metrics, multiple lenses can be applied that are guided by two tuning distinct parameters to balance the network—resolution and gain. While resolution modulates the overlapping bins (or nodes) as identified by clustering, gain controls the overlap between these bins. Nodes that do not contain shared data sample with others depending on the metric and/or resolution and gain, some may remain singleton.

In the present study, numeric data were applied in the generation of the model, thus Norm Correlation (Equation 1 shown below) was selected—a metric that measure numerical data point. The metric normalizes the features selected for generating models to have mean 0 and variance 1 and calculates Pearson's Correlation on the data.

$$NormCorr ( X , Y ) = 1 - r ( {X}^{′} , {Y}^{′} )$$

where X′, Y′ are the mean-centered and variance-normalized X and Y:

$$r ( X , Y ) = \frac{N {\sum }_{i = 1}^{N} {X}_{i} {Y}_{i} - {\sum }_{i = 1}^{N} {X}_{i} {\sum }_{i = 1}^{N} {Y}_{i}}{\sqrt{n {\sum }_{i} {X}_{i}^{2} - {( {\sum }_{i} {X}_{i} )}^{2}} \sqrt{N {\sum }_{i} {Y}_{i}^{2} - {( {\sum }_{i} {Y}_{i} )}^{2}}}$$

### Equation 1 Normalized Correlation

Furthermore, TDA utilizes lenses that summarize and separate pertinent information from the noisy data. However, each function that is selected as lenses summarize data diversely. In the present study, multidimensional scaling lenses (both resolution: 30, gain: 3.0, equalized) were applied to the dataset for generating the network. Once the TDA network was generated, the overlaying colors on the network demonstrated typical patients with certain characteristics of the variable chosen such as clinical outcomes in the evaluation of the network

### Reproducibility of Tissue Texture

Top texture-based tissue features for predicting myocardial fibrosis were defined using the top importance gain of the four regions. Variability related to the operator, device settings, and device vendors used were assessed in the study. The interobserver variability of feature extraction was tested in two blinded observers who independently analyzed twenty randomly selected patients and assessed the consistency of the texture features. To test the resistance of the texture features to device settings, changes of texture features were evaluated in different gain settings and image qualities. After adding five levels of gain (to I(x)+20, I(x)+40, I(x)+60, I(x)+80, I(x)+100) and Gaussian additive noise (mean=0, variance of 0.01, 0.02, 0.03, 0.04, and 0.05) to ten images using MATLAB R2018a (The MathWorks, Natick, MA, USA), the texture features were extracted using exactly the same ROIs. Lastly, the vendor dependency of the texture features were verified by testing topological data analysis-based patient similarity networks generated using features extracted from two vendors (GE Healthcare and Hitachi Healthcare).

### Statistical Analysis

Data are presented as the median [1st and 3rd interquartile range] for continuous variables and as the frequency (%) for categorical variables. Group differences were evaluated using Mann-Whitney U tests for continuous variables and chi-square or Fisher's exact tests for categorical variables. Kaplan-Meier curve analysis, the log-rank test, and multivariable Cox proportional hazard models were used for survival analysis. The ROC curves of the machine learning models were drawn, and the best thresholds were identified based on the Youden index. Interobserver variability was evaluated using Pearson's r and interclass correlation coefficients. All statistical analyses were performed with R version 3.5.2 (The R Foundation for Statistical Computing, Vienna, Austria). A two-tailed p<0.05 indicated statistical significance.

### Results

### Unsupervised Phenotyping Based on Texture Features

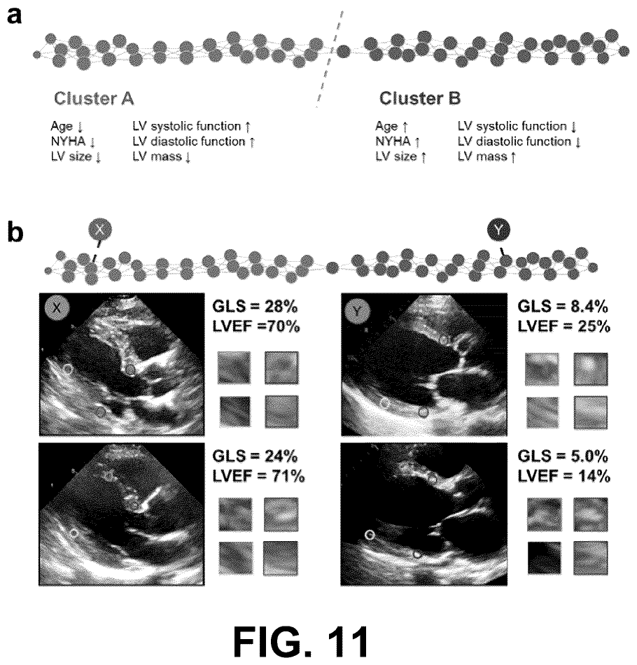

For the first part of the study (FIG. 10 (a)), 328 texture features were successfully extracted from still-frame ultrasound images in 392 of the 405 (97%) subjects. Overall, the median age of the population was 58 [45_68] years, 55.9% were female, 26.3% had severe heart failure symptoms (NYHA class III or IV) and 32.9% had stage C or D heart failure. Using the extracted texture features, unsupervised topological data analysis identified a bar-shaped patient similarity network, where two clusters were connected by a single node (FIG. 11(a)). Clusters A and B included 196 and 210 patients, respectively, with fourteen (14) patients overlapping between the groups. Interestingly, these identified clusters had significantly different clinical and echocardiographic characteristics even though the clusters were created using only the texture features of still images. Group differences are summarized in Table 1.

TABLE 1
Patient characteristics.

| Factor | Overall | | Cluster A | | Cluster B | | p value |
|---|---|---|---|---|---|---|---|
| Number of patients | 392 | | 196 | | 210 | | |
| Age, years | 58 | [45-68] | 54 | [40-65] | 61 | [50-71] | <0.001 |
| Female, n (%) | 219 | (55.9) | 113 | (57.7) | 113 | (53.8) | 0.484 |
| BSA, m² | 2.02 | [1.8-2.22] | 2.03 | [1.84-2.23] | 1.97 | [1.77-2.20] | 0.041 |
| BMI, kg/m² | 29.2 | [25.7-36.0] | 29.9 | [25.7-37.4] | 28.8 | [25.7-33.8] | 0.113 |
| Coronary artery disease, n (%) | 148 | (37.8) | 63 | (32.1) | 90 | (42.9) | 0.031 |
| Hypertension, n (%) | 272 | (69.4) | 133 | (67.9) | 148 | (70.5) | 0.592 |
| Cerebral vessel disease, n (%) | 46 | (11.8) | 21 | (10.7) | 25 | (12.0) | 0.755 |
| Diabetes mellitus, n (%) | 119 | (30.4) | 50 | (25.5) | 72 | (34.3) | 0.065 |
| Atrial fibrillation, n (%) | 50 | (12.8) | 12 | (6.1) | 42 | (20.0) | <0.001 |
| NYHA ≥ III, n (%) | 103 | (26.3) | 37 | (18.9) | 73 | (34.8) | <0.001 |
| HF stages C or D, n (%) | 129 | (32.9) | 56 | (28.6) | 78 | (37.1) | 0.073 |
| Echocardiography | | | | | | | |
| IVSd, mm | 10 | [9-12] | 10 | [8-12] | 11 | [9-13] | 0.005 |
| PWd, mm | 9 | [8-11] | 9 | [8-10] | 9 | [8-11] | 0.068 |
| LVIDd, mm | 46 | [42-51] | 46 | [42-49] | 47 | [42-52] | 0.036 |
| LVIDs, mm | 32 | [28-37] | 31 | [28-35] | 34 | [29-40] | <0.001 |
| LVEDVi, mL/m² | 52 | [43-64] | 50 | [41-60] | 56 | [45-68] | <0.001 |
| LVESVi, mL/m² | 21 | [15-28] | 19 | [15-26] | 22 | [16-33] | <0.001 |
| LA volume index, mL/m² | 25 | [20-35] | 23 | [18-32] | 28 | [21-41] | <0.001 |
| LV mass index, mg/m2 | 76 | [59-101] | 72 | [56-90] | 82 | [64-112] | <0.001 |
| E wave velocity, m/s | 0.84 | [0.69-0.96] | 0.81 | [0.69-0.94] | 0.87 | [0.70-1.01] | 0.057 |
| A wave velocity, m/s | 0.70 | [0.54-0.89] | 0.70 | [0.53-0.89] | 0.70 | [0.55-0.90] | 0.897 |
| E/A ratio | 1.14 | [0.85-1.55] | 1.11 | [0.85-1.51] | 1.16 | [0.84-1.56] | 0.720 |
| e′, cm/s | 8.1 | [6.0-10.5] | 8.5 | [7.0-10.7] | 7.6 | [5.5-10.5] | 0.001 |
| E/e′ | 9.3 | [7.2-14.5] | 9.0 | [7.2-12] | 10.1 | [7.7-17.2] | 0.003 |
| LVEF, % | 60 | [53-65] | 61 | [55-65] | 59 | [46-65] | 0.011 |
| GLS, absolute % | 19.3 | [15.7-22.0] | 20.2 | [18.1-23.5] | 17.3 | [12.5-20.4] | <0.001 |
| LV hypertrophy, n (%) | 84 | (21.4) | 23 | (11.7) | 63 | (30.0) | <0.001 |
| LV diastolic function, n (%) | | | | | | | <0.001 |
| normal | 188 | (49.3) | 120 | (61.9) | 74 | (36.8) | |
| grade 1 | 29 | (7.6) | 10 | (5.2) | 20 | (10.0) | |
| grade 2 | 63 | (16.5) | 22 | (11.3) | 45 | (22.4) | |
| grade 3 | 34 | (8.9) | 8 | (4.1) | 27 | (13.4) | |
| indeterminate | 61 | (16.0) | 31 | (16.0) | 32 | (15.9) | |
| indeterminate grade | 6 | (1.6) | 3 | (1.5) | 3 | (1.5) | |

Compared with cluster A, cluster B was associated with greater age and more advanced heart failure. Furthermore, patients in cluster B had significant differences in LV remodeling: the intraventricular septum and LV mass index were greater, the LV dimensions and volumes were larger, the LVEF and LV GLS were reduced, the LV diastolic function represented by tissue Doppler e′ and E/e′ was impaired, and the left atrial volume was larger compared to those in cluster A (Table 1). Illustrative cases for each cluster are shown in FIG. 11(b). Although functional evaluation of the myocardial textures in the images seems unfeasible with the human eye, this texture-based approach was able to identify important information for evaluating cardiac function from still routine ultrasound images and classified patients in a clinically meaningful way.

### Comparison of Clinical Outcomes Between Clusters

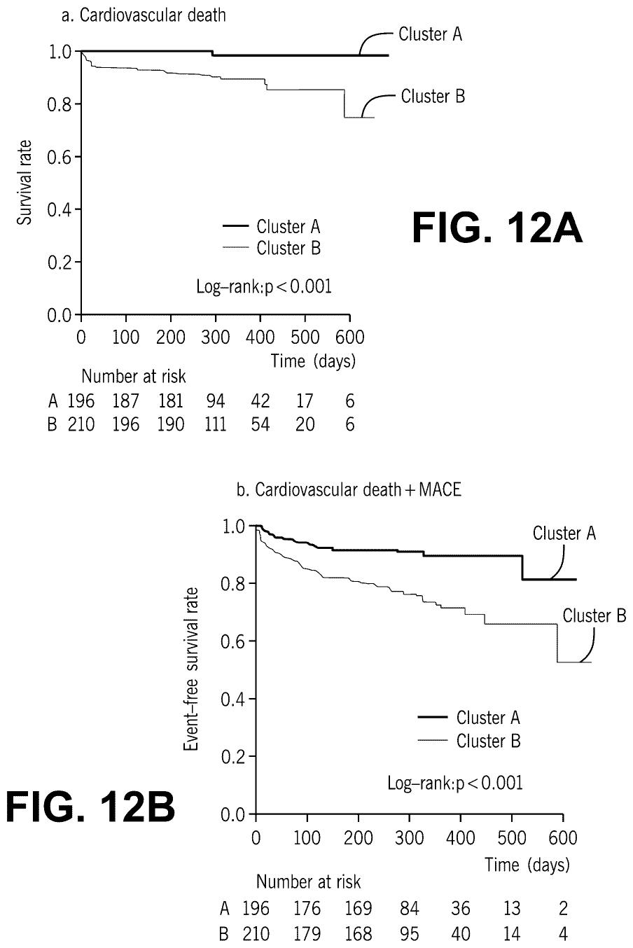

The clinical prognosis of the two clusters were also compared. During the follow-up period of a median of 301 [268-323] days, 76 MACEs, including 26 cardiac deaths, were observed. Kaplan-Meier curves showed that patients in cluster B had a significantly higher incidence of cardiac death and MACE than those in cluster A (p<0.001 by log-rank test, for both, FIGS. 12A-12B). Cox proportional hazard models showed that even after adjusting for the MAGGIC score, a well-established risk score for patients with heart failure validated in various clinical settings, texture-based clustering was significantly associated with cardiac death (HR 6.23, 95% CI 1.46-26.5, p=0.013 by Cox proportional hazard analysis). The association of texture-based clustering with MACE was significant in the univariate model (HR 2.85, 95% CI 1.69-4.78, p<0.001 by Cox proportional hazard analysis) and a model adjusted with other clinical factors (age, sex, body mass index, history of coronary heart disease, hypertension, and LVEF; HR 1.74, 95% CI 1.01-3.00, p=0.047 by Cox proportional hazard analysis), and showed borderline significance in a model adjusted with the MAGGIC score (HR 1.68, 95% CI 0.99-2.87, p=0.057 by Cox proportional hazard analysis). The results for the Cox models are summarized in Table 2.

TABLE 2
Cox regression models.

| | For cardiac death | | | For MACE | | |
|---|---|---|---|---|---|---|
| | | | p | | | p |
| | HR | 95% CI | value | HR | 95% CI | value |
| Cluster B | 11.09 | 2.62-46.93 | 0.001 | 2.85 | 1.69-4.78 | <0.001 |
| Adjustment for | 6.23 | 1.46-26.50 | 0.013 | 1.68 | 0.99-2.87 | 0.057 |
| MAGGIC score | | | | | | |
| Adjustment for | | | | 1.74 | 1.01-3.00 | 0.047 |
| clinical factors* | | | | | | |

### Supervised Learning-Based Prediction of Functional LV Remodeling

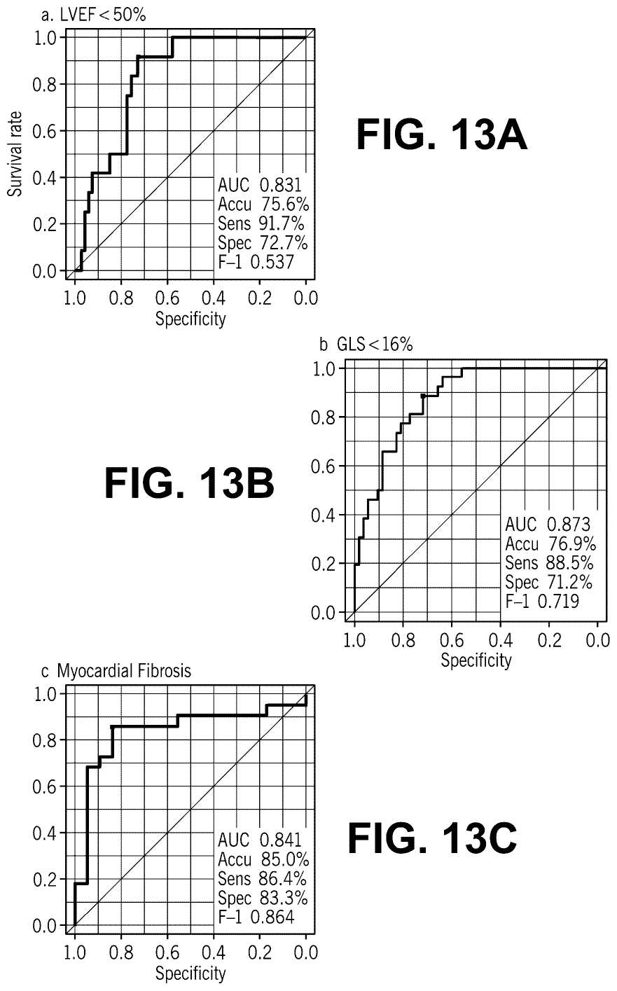

This part is summarized in the bottom left of FIG. 10. To explore the value of the texture-based tissue features to directly predict functional LV remodeling, the patient cohort was randomly divided into training (80%) and test set (20%), and supervised machine learning algorithms were trained in the training set using only the texture features extracted from the still images. Topological data analysis and subsequent recursive feature elimination were used for feature selection and 18 features per each were used to train prediction models for reduced LVEF (<50%) and GLS (<16%). Panel A and B in FIGS. 13A-13C show the ROC curves for the prediction of reduced LVEF and GLS obtained in the test set. The best model for predicting reduced LVEF was an ensemble of 10 models (2 boosted trees, 3 random decision forests, 2 LASSO regressions, 2 ridge regressions, and 1 neural network) and showed performance of ROC AUC 0.83, sensitivity 91.7%, and specificity 72.3%, whereas the one for impaired GLS was an ensemble of 10 different models (5 bootstrap decision forest, 2 ridge regressions, 2 LASSO regressions, and 1 neural network) and had ROC AUC of 0.87, sensitivity of 88.5%, and specificity of 71.2%.

### Supervised Learning-Based Prediction of Myocardial Fibrosis

For investigating the value of the cardiac ultrasound texture-based features in predicting whether the patient has myocardial fibrosis detected by CMR, 89 retrospectively identified patients who had undergone CMR and echocardiography within 48 hours were studied as the training set, and 40 independent prospective patients were studied as the test set, as shown in FIG. 10 (b). Texture feature extraction was feasible in 85 (96%) and 40 (100%) patients, respectively. The clinical characteristics of the training and test set are summarized in Table 3.

TABLE 3
Patient characteristics of the patients with a magnetic resonance scan.

| | Retrospective | | Prospective | | |
|---|---|---|---|---|---|
| Factor | (training) | | (rest) | | p value |
| Number of patients | 85 | | 40 | | |
| Age, years | 55 | [41-66] | 56 | [46-64] | 0.667 |
| Female, n (%) | 53 | (62.4) | 16 | (40.0) | 0.022 |
| BSA, m² | 2.03 | (0.35) | 2.03 | (0.27) | 0.984 |
| BMI, kg/m² | 55.37 | (231.98) | 29.77 | (6.23) | 0.488 |
| Coronary artery disease, n (%) | 38 | (44.7) | 13 | (33.3) | 0.246 |
| Hypertension, n (%) | 43 | (50.6) | 27 | (69.2) | 0.078 |
| Cerebral vessel disease, n (%) | 6 | (7.1) | 3 | (12.5) | 0.410 |
| Diabetes mellitus, n (%) | 21 | (24.7) | 13 | (33.3) | 0.387 |
| Atrial fibrillation, n (%) | 8 | (9.4) | 4 | (10.3) | >0.99 |
| NYHA ≥ III, n (%) | 12 | (14.1) | 16 | (40.0) | 0.002 |
| HF stages C or D, n (%) | 17 | (20.0) | 21 | (52.5) | <0.001 |
| Echocardiography | | | | | |
| IVSd, mm | 10 | [8-12] | 10 | [9-11] | 0.983 |
| PWd, mm | 9 | [8-11] | 10 | [9-11] | 0.503 |
| LVIDd, mm | 48 | [42-53] | 46 | [45-57] | 0.163 |
| LVIDs, mm | 34 | [29-42] | 35 | [30-48] | 0.378 |
| LVEDVi, mL/m² | 54 | [40-67] | 57 | [45-69] | 0.537 |
| LVESVi, mL/m² | 23 | [16-40] | 27 | [20-45] | 0.168 |
| LA volume index, mL/m³ | 22 | [18-33] | 32 | [21-39] | 0.011 |
| LV mass index, mg/m2 | 79 | [67-103] | 96 | [70-132] | 0.057 |
| E wave velocity, m/s | 0.85 | [0.62-1.00] | 0.86 | [0.69-1.00] | 0.816 |
| A wave velocity, m/s | 0.60 | [0.51-0.84] | 0.68 | [0.49-0.83] | 0.980 |
| E/A ratio | 1.26 | [0.84-1.77] | 1.16 | [0.90-1.66] | 0.810 |
| e′, cm/s | 9 | [6-12] | 8 | [6-11] | 0.317 |
| E/e′ | 8.4 | [6.6-11.3] | 10.4 | [7.6-14.8] | 0.093 |
| LVEF, % | 55 | [42-63] | 48 | [34-56] | 0.023 |
| GLS, absolute % | NA | | 12.8 | [7.9-19.4] | NA |
| LV hypertrophy, n (%) | | | | | 0.136 |
| LV diastolic function, n (%) | | | | | 0.173 |
| Normal | 17 | (20.0) | 3 | (8.3) | |
| Grade 1 | 28 | (32.9) | 11 | (30.6) | |
| Grade 2 | 18 | (21.2) | 15 | (41.7) | |
| Grade 3 | 17 | (20.0) | 5 | (13.9) | |
| Indeterminate | 5 | (5.9) | 2 | (5.6) | |
| Indeterminate grade | 0 | (0.0) | 0 | (0.0) | |

There were 48 (56.4%) and 22 (55.0%) patients who had myocardial fibrosis in at least one of the four ROIs where the texture features were extracted using the ultrasound images in the training and test set, respectively.

Out of the extracted texture features, feature selection was performed and five best features were identified to develop supervised machine learning models. The models were trained to predict whether the patient has myocardial fibrosis using cross-validation in the training set (FIGS. 5A-5C). In the test set, the developed model (an ensemble of 2 LASSO regressions) predicted myocardial fibrosis with an ROC AUC of 0.84 (sensitivity 86.4%, and specificity 83.3%).

### Robustness of Texture-Based Feature Extraction

To confirm the stability of the texture features, the variability related to the operator, image quality, and device vendors used in the study were accessed. Briefly, most features had a good interobserver agreement with interclass correlation coefficient 0.74-0.96, except for correlation in GLCM (0.54). The noise and gain on each image were artificially increased and the change of the features was tested. As shown, each feature showed different behavior against increases in gain and noise. For example, gray-level non-uniformity of the GLRLM, the second most important feature for predicting functional LV remodeling, was resistant to an increase in gain, while it markedly increased with additional noise in the images. On the other hand, the high gray-level run emphasis of GLCM, one of the important features for predicting myocardial fibrosis, was relatively resistant to an increase in noise, whereas it dramatically increased with additional gains on the images. To elucidate the value of the texture features among different vendors, patient similarity networks were created using data obtained from each of the two dominant vendors using topological data analysis. The created networks from both vendors formed similar loops, where most patients with reduced LV systolic function were segregated in a part of a loop, suggesting that the information content of the texture features were relatively invariant to the data source.

### DISCUSSION

In the present study, the texture-based analysis was illustrated to be feasible for most clinical cardiac ultrasound (97%), unsupervised patient-similarity analysis revealed that a specific pattern of information from myocardial texture was associated with functional LV remodeling, advanced heart failure, and adverse clinical outcome, and the texture features extracted from still cardiac ultrasound images could be used for developing supervised machine learning models that enable clinical prediction of functional and structural LV remodeling. Texture-based analysis has been recently used in radiology (also referred to as radiomics) to extract maximal information from standard-of-care images using high-throughput computing. The present disclosure resembles the general principles of radiomics and specifically defines a computational pipeline where texture-based tissue features were extracted and used for building supervised machine learning models for individualized predictions. This approach may potentially address a long-described objective of cardiac ultrasound in providing myocardial tissue characterization in clinical practice.

It is well known that in typical cases, the pathological myocardial texture is visually distinguishable with ultrasound images. For example, scar lesions after myocardial infarction have high echo intensity and thin walls, and myocardium with infiltration of amyloid has a granular sparkling texture. However, many previous attempts to characterize myocardial tissue using ultrasound images, such as integrated backscatter analysis, have resulted in suboptimal results because of variations in the quality and texture of the cardiac ultrasound images. As a consequence, in current clinical practice, CMR imaging is preferred modality for myocardial tissue characterization using late gadolinium enhancement imaging and methods such as parametric and non-parametric T1, T2 and T2* imaging. However, due to its cost, accessibility, and contraindications, CMR is not available for every patient and in every place. Since cardiac ultrasound remains portable, low-cost, and the most common cardiac imaging procedure performed in clinical practice, implementation of tissue characterization with cardiac ultrasound may have a wider clinical impact. In the initial attempt, the use of cardiac ultrasound texture-based tissue features of myocardium were illustrated to be robust and concordant in several steps of analyses: i.e., cluster analysis with topological data analysis with clinical outcome prediction; supervised machine learning analysis for predicting impaired LV systolic function; and identification of the presence of myocardial fibrosis.

The segregation of high-risk myocardium was also shown to be vendor-independent and that interobserver agreement was adequate for clinical application. These results reconfirmed that important information associated with myocardial remodeling that can be captured by CMR is also carried in ultrasound texture features and can be retrieved using a modern high-throughput computing pipeline, which possibly amended the signal to noise ratio and helped extraction of useful information from noisy ultrasound data.

Although some features were sensitive to changes in gain or noise, the majority of the features were stable and resistant to the changes in image quality. Although radiomics-based texture analysis approach in this study, deep learning may be another choice of approaches with which images can be analyzed in an end-to-end pipeline. Both deep learning and radiomics have received considerable attention in recent years in radiology and the relative merits of both techniques remains an area of active investigation. While some investigators have only recently compared the two approaches citing the advantage of deep learning approaches for radiological images, others have suggested that both approaches are complementary and can unite in the future to produce a single unified framework. Such comparative studies have been performed mostly in radiology, in general, and the application of radiomics for cardiac imaging is still in its infancy.

The embodiments of the present disclosure are novel with respect to the application of traditional radiomics approach to extract semantic and agnostic features from cardiac ultrasound images for predicting LV remodeling. A recent successful application of handcrafted radiomics features in myocardial tissue characterization further supports the choice of restricting the initial analysis to only using handcrafted radiomics approach. While deep learning based radiomics may have several advantages including its generalization capability and its independence from the supervision of experts, the lack of reproducibility and interpretability, as well as over-fitting on small datasets like those of the present disclosure, pose substantial challenges in readily adapting deep networks for this study.

### Experiment 3

Acute myocardial infarction (MI) is one of the leading causes of morbidity and mortality globally. The prevalence of the disease approaches 3 million people worldwide, with 605,000 new MIs diagnosed annually in the US. Echocardiography is a rapid, noninvasive, portable, and inexpensive imaging modality, making it the preferred technique for assessing MI patients. Although echocardiography visualizes the effects of ischemia and MI on regional and global myocardial function, direct identification and quantification of infarcted tissue remains challenging and has not been extensively explored, especially when compared to cardiac magnetic resonance (CMR) imaging, which is considered the gold standard for infarct tissue characterization.

Among other aspects, Experiment 3 compares the diagnostic value of handcrafted ultrasound radiomics (extracted from echocardiography images using predefined mathematical algorithms) with deep learning-derived features (generated through automated methods like convolutional neural networks) for distinguishing patients with and without acute MI, describes the independent and incremental value of ultrasound radiomics over conventional echocardiographic parameters including longitudinal strain for detecting acute MI, demonstrates the feasibility of ultrasound radiomic features to localize infarcted tissue and create a parametric map of infarcted myocardium using paired CMR assessments, and demonstrates post-MI infarct size quantification—an important predictor of mortality and a key endpoint for MI cardioprotection strategies.

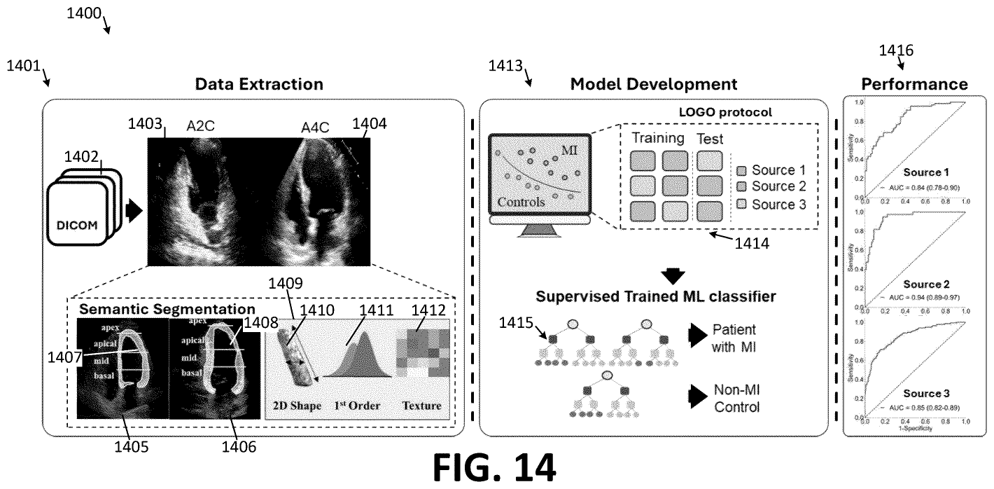

FIG. 14 illustrates a block diagram of an ultrasound radiomics system 1400. The system comprises three main sections: data extraction 1401, model development 1413, and performance analysis 1416.

The data extraction section 1401 begins with DICOM images 1402 as input. These images may include, for example, an apical two chamber view 1403 and an apical four chamber view 1404 of the heart. The DICOM images 1402 may be acquired using standard ultrasound equipment from vendors such as GE Healthcare, Philips Healthcare, or Hitachi Healthcare. Other examples of cardiac views that may be used include parasternal long-axis views, parasternal short-axis views, subcostal views, and suprasternal views. The apical two chamber view 1403 provides visualization of the left ventricle and left atrium, while the apical four chamber view 1404 displays all four cardiac chambers simultaneously. The system processes these views to generate segmented images 1405 and 1406, which are further divided into segments 1407 and 1408. The segmentation process may utilize automated algorithms that delineate the left ventricular myocardium throughout the cardiac cycle from end-diastole to end-systole, creating binary masks that mark specific myocardium regions within the echocardiographic images. In some examples, this segmentation process may be an implementation of the image analysis techniques described in FIG. 1, where basic statistics and spatial resampling variables 106 are extracted from ultrasound images 103.

A feature identification component 1409 analyzes the segmented images to extract multiple types of features: shape feature 1410, first order feature 1411, and texture feature 1412. The shape feature 1410 may include metrics such as myocardial wall thickness, chamber dimensions, and contour characteristics. The first order feature 1411 may comprise statistical measures like mean intensity, standard deviation, skewness, and kurtosis of pixel values within the segmented regions. The texture feature 1412 may include more complex patterns derived from matrices such as Gray Level Co-occurrence Matrix (GLCM), Gray Level Zone Length Matrix (GLZLM), Gray Level Run Length Matrix (GLRLM), and Neighborhood Grey Level Different Matrix (NGLDM). For example, this feature extraction process may correspond to the texture features 112 identified in FIG. 1. For instance, the feature extraction could be implemented using open-source software libraries such as PyRadiomics, SimpleITK (e.g., within a Python framework).

The model development section 1413 incorporates a training protocol 1414 that processes the extracted features. The training protocol 1414 may implement a leave-one-source-out cross-validation (LOSO-CV) strategy to evaluate model performance across multiple data sources, keeping all data from the same group together in either training or testing sets. This approach helps assess generalizability across diverse patient populations. The protocol may also include feature selection techniques such as recursive feature elimination to identify the most relevant features for model development. In some cases, this training protocol may be an implementation of the machine learning models described in step 809 of FIG. 8. The training protocol 1414 feeds into a classifier network 1415, which analyzes the processed data. The classifier network 1415 may be implemented as an ensemble of multiple models including gradient boosting machines (like XGBoost), random forests, LASSO regressions, ridge regressions, and neural networks to improve prediction accuracy. The classifier could be designed to distinguish between patients with myocardial infarction and non-MI controls based on the extracted radiomic features. As an example, this classifier network may be similar to the neural network classifier or deep learning classifier mentioned in FIG. 7.

The system outputs results through a performance graph 1416, which displays the analysis outcomes and system performance metrics. The performance graph 1416 may include receiver operating characteristic (ROC) curves showing the relationship between sensitivity and specificity, with area under the curve (AUC) values quantifying overall model performance. The graph could also display other metrics such as accuracy, F1-score, positive likelihood ratio, and negative likelihood ratio. The visualization may be implemented using data visualization libraries such as Matplotlib or Plotly in Python. In some instances, this performance evaluation may correspond to the clinical significance determination described in step 812 of FIG. 8.

The overall structure of the ultrasound radiomics system 1400 in FIG. 14 may be implemented on computing hardware similar to the computing device 900 described in FIG. 9. The system could utilize a processor 903 such as a multi-core CPU or GPU for parallel processing of image data, with sufficient memory 906 to handle the computational demands of feature extraction and model training. The system might employ a distributed architecture where image processing and feature extraction occur on edge devices near the ultrasound equipment, while model training and evaluation are performed on centralized servers with greater computational resources. For example, the various components of the system 1400 could be implemented as software modules within the myocardial ultrasonic fingerprinting application 915 stored in memory 906 and executed by processor 903.

The segmentation process shown in FIG. 14 (segmented images 1405, 1406 and segments 1407, 1408) may utilize deep learning-based segmentation networks such as U-Net or V-Net architectures trained on manually annotated cardiac ultrasound images. The segmentation algorithm could automatically identify the endocardial and epicardial borders of the left ventricle, dividing the myocardium into the conventional 17-segment model defined by the American Heart Association. The algorithm might incorporate temporal information across the cardiac cycle to improve segmentation accuracy. In some cases, this segmentation process may be used to identify regions of interest similar to those described in FIGS. 5A-5C, where normal myocardium images 512a and infarcted myocardium images 512b are analyzed.

The classifier network 1415 in FIG. 14 may implement unsupervised clustering techniques such as topological data analysis (TDA) to identify patient groups with similar radiomic features. The network could apply metrics like Norm Correlation to measure numerical data points, normalizing features to have mean 0 and variance 1 before calculating Pearson's Correlation. The clustering algorithm might utilize multidimensional scaling lenses with specific resolution and gain parameters (e.g., resolution: 30, gain: 3.0, equalized) to generate the network. In some examples, the network could use the extracted features to group patients into clusters similar to the circular network cluster 203a or linear network cluster 203b shown in FIGS. 2A and 2B.

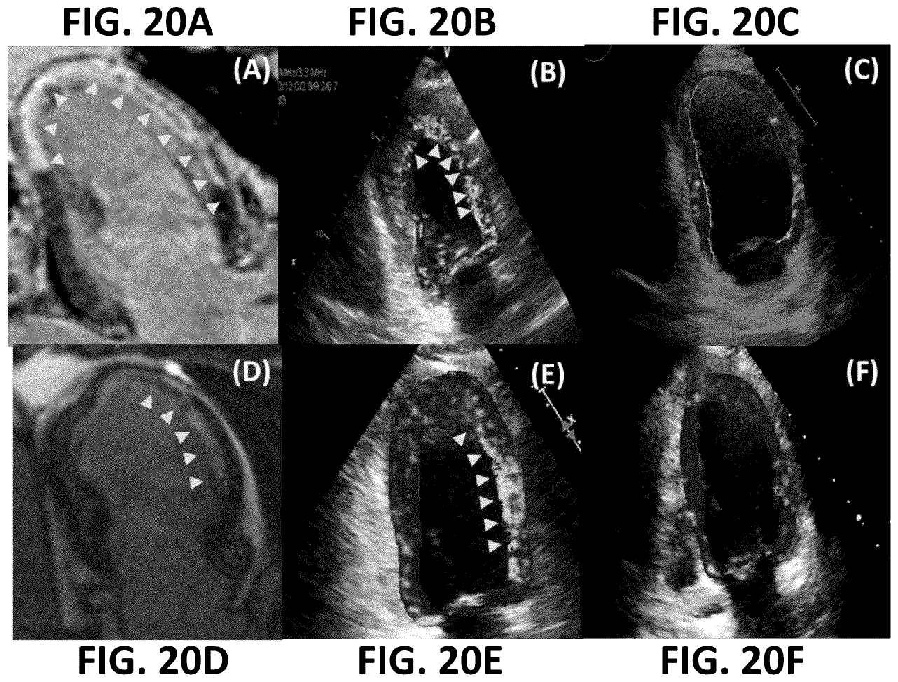

The performance graph 1416 in FIG. 14 may include interactive visualization techniques that allow clinicians to explore the relationship between different radiomic features and clinical outcomes. The graph could include scatter plots, bar charts, and survival curves that illustrate how different texture patterns correlate with cardiac function metrics like global longitudinal strain (GLS) and left ventricular ejection fraction (LVEF). The visualization might incorporate color coding to highlight statistically significant differences between patient groups. As an example, the performance graph may present results in a format similar to the texture-based phenotyping comparison shown in FIG. 3, where different textures are associated with varying clinical outcomes. When applied to a specific patient, the system could generate visual outputs including color-coded parametric maps overlaid on the original ultrasound images, similar to those shown in FIGS. 20A-20F, where blue overlays highlight regions of potential infarction. The system could also produce segmental bull's-eye plots showing the distribution of radiomic features across different myocardial segments, quantitative infarct size measurements expressed as a percentage of total myocardial volume, and comprehensive clinical reports integrating radiomic findings with conventional echocardiographic parameters. These reports might include risk stratification scores based on the patient's radiomic signature compared to established patient clusters as shown in FIG. 6, with clear visual indicators distinguishing high-risk from low-risk myocardial characteristics.

The feature extraction and classification processes in FIG. 14 may implement ComBat harmonization techniques to reduce scanner-related variability and batch effects in the radiomics data. This harmonization process could standardize features across different ultrasound equipment vendors, improving model generalizability. The system might incorporate sensitivity analysis to assess the robustness of radiomic features to noise and altered image gains, applying multiple levels of Gaussian noise (mean=0, variances 0.01 to 0.05) and gain adjustments to test feature stability. In some instances, the shape features 1410, first order features 1411, and texture features 1412 extracted in FIG. 14 could be used to differentiate between high-risk myocardium 603 and low-risk myocardium 606 as depicted in FIG. 6, with specific features like gray-level non-uniformity from the gray-level dependence matrix serving as discriminative markers for infarcted versus normal myocardium.

### Methods

### Clinical Population and Study Design

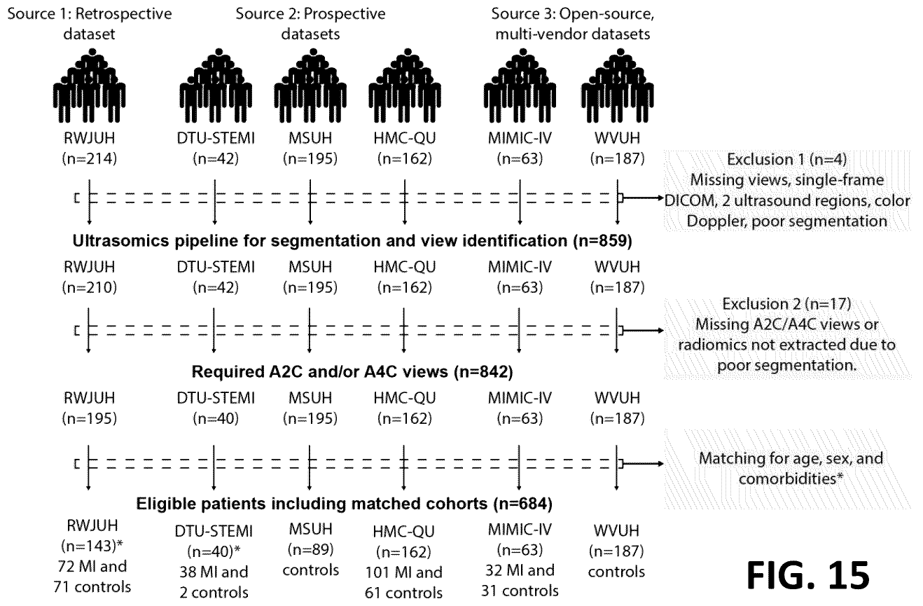

A total of 684 subjects from six sites were divided into three data sources: A) retrospective single-center matched case-controls (Data Source A), B) prospective multicenter matched clinical trial dataset (Data Source B), and C) open-source unmatched international and multivendor dataset (Data Source C) (FIG. 15).

The retrospective single-center matched case-control cohort (Data Source A) comprised 143 subjects (72 MI and 71 non-MI controls) admitted between January 2023 and December 2024 at RWJUH and matched for age, sex, and underlying comorbidities.

The prospective clinical trial dataset (Data Source B) comprised 40 participants from the DTU-STEMI (Door-To-Unload in STEMI) trial-a prospective, multicenter, randomized pilot trial involving 14 centers in the United States and 89 matched non-MI controls enrolled between March 2013 and December 2015 from the Mount Sinai University Hospital (MSUH) in a clinical study where subjects underwent echocardiography and computed tomography coronary angiography for exploring the development of machine learning models of diastolic dysfunction from surface ECG.

The open-source international dataset (Data Source C) comprised 162 individuals (101 MI cases, 61 non-MI controls) collected between 2018 and 2019 from Hamad Medical College-Qatar University (HMC-QU), and 32 participants with MI from the Medical Information Mart for Intensive Care IV with Echocardiogram (MIMIC-IV-ECHO), who were admitted between 2017 and 2019. This dataset was enriched with controls with varying age and risk exposures to understand how ultrasound radiomic features associate with MI independent of age and risk factors. This included 31 non-MI older controls and a multivendor database (Table 4) comprising 187 non-MI controls (57 healthy and 130 with cardiovascular risk factors) collected between July 2017 and February 2018 from West Virginia University Hospital (WVUH).

TABLE 4
Image processing attributes from diverse databases.

| | Source A: | | | | | |
|---|---|---|---|---|---|---|
| | Retrospective | | | | | |
| | trial | Source B: Prospective | | Source C: Open-source and multi- | | |
| Attributes | (n = 143) | Trial ( n = 129 ) | | vendor Databases (n = 412) | | |
| Sites | RWJUH | DTU- | MSUH | HMC-QU | MIMIC- | WVUH |
| | (n = 143) | STEMI | (n = 89 ) | (n = 162 ) | IVECHO | (187) |
| | | n = 40) | | | (n = 63 ) | |
| Timelines | January | (August | August | 2018 to | 2017 to | August |
| | 2023 to July | 2017 to | 2017 to | 2019 | 2019 | 2017 to |
| | 2023 | Septemb | Septemb | | | Septemb |
| | | er 2018 | er 2018 | | | er 2018 |
| Vendors | GE (Vivid- | GE (Vivid- | GE (Vivid- | Phillips | GE (Vivid- | GE (Vivid- |
| | 9/95) | 9/95) | 9/95) | and GE | E90, E95, | 9/95), |
| | | | | Vivid | and S7) | and |
| | | | | | | Hitachi |
| | | | | | | (LISENDO |
| | | | | | | 880) |
| Average | 30 | 25 | 50 | 25 | 31 | 30 |
| frame | | | | | | |
| rate of | | | | | | |
| the | | | | | | |
| converte | | | | | | |
| d DICOM | | | | | | |
| Average | | | | | | |
| frames | 13 | 13 | 23 | 17 | 13-15 | 13 |
| within a | | | | | | |
| cardiac | | | | | | |
| cycle | | | | | | |
| Spatial | | | | | | |
| Resolution | 1024×1024 | 1024×10 | 1024×10 | 1024×10 | 1024×10 | 1024×10 |
| Post | pixels | 24 pixels | 24 pixels | 24 pixels | 24 pixels | 24 pixels |

RWJUH = Robert Wood Johnson University Hospital,
DTU-STEMI = Door-to-unload
STEMI, MSUH = Mount Sinai University Hospital,
HMC-QU = Hamad Medical College, Qatar University,
MIMIC-IV-ECHO = Medical Information Mart for Intensive Care IV with Echocardiogram,
WVUH = West Virginia University Hospital,
GE = General Electric

All databases were approved by the local IRBs. Participants in the prospective cohorts provided written informed consent. The studies adhered to institutional and national ethical standards and the 1964 Helsinki Declaration.

### Defining the Status of Myocardial Infarction

Data Source A: Patients with ST-elevation MI (STEMI) in the RWJUH dataset were identified using clinical findings, electrocardiographic changes, and biomarker levels according to the current universal definition of MI. STEMI was classified per the Joint ESC/ACCF/AHA/WHF Task Force. Briefly, this included ECG changes revealing 1) new ST-segment elevation in 2 contiguous leads with greater than 0.1 mV in all leads, with the exception of V2 or V3, 2) new ST-segment elevation in leads V2-V3 greater than 0.2 mV (men>40 years old), 0.25 mV (men<40 years old), or 0.15 mV (women), 3) Pre-existing left bundle branch block were further evaluated using the Sgarbossa's criteria. Exclusion criteria included (1) patients discharged to institutionalized care, (2) type 2-5 acute myocardial infarction (AMI), (3) co-existing terminal illness such as cancer, (4) alternative diagnosis for elevated cardiac troponin values (e.g. myocarditis, pericarditis, non-ischemic cardiomyopathies, moderate-severe valvular heart disease), (5) pregnancy, and (6) technically insufficient imaging for 2 of the following 3 views: apical 4chamber (A4C), apical 3-chamber (A3C), and 2-chamber (A2C).

Data Source B: In the DTU-STEMI trial, patients underwent CMR imaging on days 3 to 5 and day 30 (±7 days) using standard protocol. A central core laboratory (Duke Cardiovascular Magnetic Resonance Center, Durham, NC) assessed deidentified images. The presence of an infarct in each of the 17 myocardial AHA defined echocardiography segments was labeled based on CMR-defined delayed hyperenhancement or late gadolinium enhancement (LGE) in each segment. In addition, delayed enhancement scores determined using the well-established 5-point grading system outlined by the AHA were also considered, where 0 indicates no hyperenhancement, 1 represents 1%-25%, 2 signifies 26%-50%, 3 corresponds to 51%-75%, and 4 reflects 76%-100% involvement. The CMR delayed enhancement scores were used to define segment-level infarcts (hyperenhancement score >0).

Data Source C: In the HMC-QU database, patients with STEMI were admitted and treated with coronary angiogram/angioplasty, with echocardiography performed within 24 hours or prior to intervention. Non-MI subjects were evaluated for other clinical reasons. Each myocardial segment was labeled as infarct-related or normal based on regional wall motion abnormalities. Cardiac cycle frames (end-diastole and end-systole) were defined using ECG data or by identifying frames with the largest and smallest LV areas when ECG was unavailable. In the MIMIC-IV-ECHO database, STEMI patients were identified using ICD-10 codes (I2111, I2119, I2102, I213, I2109, I2121). Control patients were selected using the ICD-10 code 110, with normal echocardiographic measurements.

### Echocardiography Image Analysis and Segmentation

All comprehensive 2D and Doppler transthoracic echocardiographic images were acquired by expert sonographers per guidelines. Average longitudinal strain (LS) was measured from 2-chamber (a2c)- and 4-chamber (a4c) views. The automated echocardiography imaging workflow for ultrasound radiomics comprises four stages: preprocessing, view identification, segmentation, and ultrasound radiomics feature extraction. The preprocessing stage converted 2D echocardiograms with varying resolutions and frame rates (Table 4) from diverse formats (.avi, .mp4) into standard DICOM format using Sante DICOM software (version 7.9.4, 64-bit) for harmonization.

DICOM files with Doppler data or dual ultrasound regions were excluded. The view identification stage classified these processed DICOM files to identify a2c and a4c transthoracic echocardiography views.

During segmentation, the left ventricular (LV) myocardium was delineated in each frame throughout a cardiac cycle (end-diastole to end-systole). A black- and white (i.e., binary) mask marked the myocardium region within the echocardiographic image for both apical views. To ensure uniformity and harmonization in ultrasound radiomic extraction, grayscale LV myocardium images and binary masks were saved at a standardized resolution of 1024×1024 pixels for all databases (Table 4).

Using an automated algorithm, the LV segments from the segmented myocardium were subsequently delineated as per the conventional AHA-defined myocardial segments for the a2c and a4c views. The apical segment (apical cap-segment 17) was excluded from the segmental analysis per guideline recommendations. Spatial resolution is maintained during segmentation to ensure each segment retains its size and relative location within the LV myocardium. The algorithm uses LV myocardial binary masks to define segments, ensuring consistent segmental positioning across all frames.

The original image and its corresponding binary myocardial segments were then fed to an ultrasound radiomics pipeline to extract 2D shape-based, 1^{st}-order, and texture based features. Ultrasound radiomic feature extraction targets 2D features from static images, not directly capturing segmental motion (e.g., wall motion abnormalities). Instead, an average of these static features across all frames represents the temporal and spectral characteristics of the cardiac cycle, providing a broader view of segmental rhythm changes. The complete automated pipeline was developed using opensource Python (version 3.7, Python Software Foundation) and has been previously validated for extracting ultrasound radiomics features.

### Morphology and Texture-Driven Handcrafted Ultrasound Radiomics

PyRadiomics (version 3.0.1, Python Software Foundation) and SimpleITK (version 2.2.0, Insight Software Consortium) employed within the open-source Python framework facilitated the extraction of ultrasound radiomics features, also referred to as handcrafted radiomics (HCR). To maintain consistency, ultrasound radiomics features were extracted from 12 segments across all databases, excluding the apical cap, as it lacks inward segmental motion activity and is therefore also not recommended in guidelines for segmental wall assessments.

A total of 98 shape, 1st-order, and texture-based features were extracted for each of the 12 myocardial segments. Root mean square and spectral entropy were calculated for each feature across all frames in a cardiac cycle to capture temporal and spectral variations. Subsequently, all the ultrasound radiomics features from a single segment from the four feature categories (98 from end-diastole, 98 from end-systole, 98 temporal, and 98 spectral) were compiled. Features from all 12 segments were gathered for each patient (totaling 2,352 features per view and 4,704 per patient) for feature engineering and machine learning model development.

In the feature engineering process, the Boruta feature selection algorithm was employed to identify the most relevant and informative features for the predictive model. Boruta is a wrapper method built around a random forest classifier, designed to capture all features that are statistically significant with respect to the target variable. Using the BorutaPy implementation in Python, features were iteratively compared in importance against randomized shadow features to determine their relevance. Features confirmed by Boruta as important were retained, ensuring that only those with strong and consistent predictive power were included in the refined training and testing datasets for model development.

### Deep Learning-Based Ultrasound Radiomics

To evaluate DTL features, four 3D-CNN-based architectures were compared: Slow-Fast R50, R(2+1)D, and a Channel-Separated Convolutional Network, and a transformer-based model. Using transfer learning, features were captured from the final classification layer of these 3D-CNN architectures. Each myocardial segment video produced 400 DTL features, resulting in 4800 features from 12 segments across a2c and a4c views per patient, used for feature engineering and ML model development. As preprocessing, each ultrasound radiomic feature was standardized (mean zero, unit variance). To address scanner-related variability and reduce batch effects in the radiomics data, the ComBat harmonization technique was applied-a widely used and robust method for correcting batch effects.

The ultrasound radiomics data was preprocessed using standardization to ensure that all features have a mean of 0 and a standard deviation of 1, bringing them to a comparable scale. This was done using the StandardScaler function from the scikit-learn library in Python. Standardization was applied by subtracting the mean and dividing by the standard deviation for each feature: z=(x−μ)/σ, where x is the original feature value, μ is the mean of the feature, and σ is the standard deviation of the feature. The StandardScaler method computes these values for each feature and transforms the data accordingly. This process ensures that each feature contributes equally to the analysis, preventing features with larger numerical ranges from dominating the model. It also accelerates the convergence of many machine-learning algorithms by improving their stability and performance. Additionally, it helps in reducing the effect of outliers, resulting in more robust model training.

### Machine Learning Model Development

The machine learning model (Central Illustration) development was performed through an automated machine learning (AutoML) pipeline (e.g., H2O.ai, version 3.44.0.2). The AutoML platform utilizes a series of ML algorithms, including generalized linear models, deep neural networks, distributed random forests, gradient boosting machines, and extreme gradient boosting.

Standard hold-out and cross-validation methods based upon random splitting of combined data can be expected to be overoptimistic when deploying models to sources not represented in the dataset. Therefore, to evaluate model performance specifically that includes patients collected across multiple sites, a leave-one-source-out cross-validation (LOSO-CV) strategy was used. Unlike standard k-fold methods that assume data points are independent, LOSO-CV keeps all data from the same group together in the training or testing set. This method of external validation has been advocated for assessing the generalizability of model performance across diverse multisite patient populations. The previously published PRIME checklist was followed for the development of ML models. The prediction probabilities served as a continuous ultrasound radiomics infarction score, with any value ≥0.5 indicating the presence of infarction and <0.5 indicating the absence of infarction.

### Performance Evaluation and Statistical Analysis

Baseline clinical characteristics are presented as median (interquartile range) for continuous variables and count (percentage) for categorical variables. Continuous variables across multiple groups are compared using a one-sample analysis of variance to see if the variable is normally distributed or if the Kruskal-Wallis test is otherwise. Categorical variables are assessed with Fisher's exact test for contingency table values below 5 or the chi-square test otherwise. Ultrasound radiomics features from infarcted and non-infarcted segments per patient across the cohort were analyzed with a paired Mann-Whitney U test, with significant features visualized in a Manhattan plot. A sensitivity analysis assessed the reproducibility and robustness of significant ultrasound radiomics features to noise and altered image gains, simulating diverse scanner settings, using the intra-class correlation coefficient (ICC). The performance of ML models was assessed using leave-one-source-out cross-validation. Predictions for each source were used to evaluate the area under the receiver operating characteristic (ROC) curve, along with other performance metrics such as sensitivity, specificity, F1 score, and accuracy. The overall performance across all three data sources was also calculated and reported as their 95% confidence interval, estimated using a bootstrapping technique. An overall ROC curve was also generated by concatenating the predictions from each held-out set. The prediction probabilities from each held-out set during the cross-validation process were used to create this curve, providing a comprehensive evaluation of the model's performance. The difference in AUCs between groups was estimated using the DeLong test. Univariate and multivariate logistic regression analyses were performed in the entire cohort using ML model predictions from each held-out group and echocardiographic features to predict MI. Variables with p-value <0.05 in univariate analysis were included in the multivariate regression model. All analyses were performed using Python (version 3.7) and MedCalc (version 12.5.0.0), with p<0.05 considered significant.

### Reproducibility of Ultrasound Radiomics and Feasibility Analysis

Ultrasound radiomics features that significantly differentiate infarcted from normal myocardium were identified using paired t-tests and Manhattan plots. Reproducibility analysis was conducted to evaluate variability in these features due to device settings and image quality. Unlike prior studies that used still frames from the parasternal long-axis view, this approach utilized ultrasound radiomics from the cardiac cycle in apical views, allowing for the capture of static and dynamic features, including temporal and spectral variations across the cardiac cycle. The robustness of these features under real-world conditions was further tested by applying five levels of gain adjustments (I(x)+20, +40, +60, +80, +100) and Gaussian noise (mean=0; variances 0.01 to 0.05) to images from 10 patients, focusing on texture features from end-diastolic LV segments across 12 segments in a2c and a4c views. To further assess the feasibility of the ultrasound radiomic ML model, the model was tested using input features perturbed by Gaussian noise with varying levels of variance (0.01, 0.03, and 0.05). The overall performance variations from the baseline optimal ML model were recorded.

### Results

### Baseline Characteristics

Tables 5-8 summarize the available baseline characteristics of the participants across Data Sources A-C, grouped according to the leave-one-source-out crossvalidation approach used in the ML model.

TABLE 5
Clinical characteristics for the cohort from source A (n = 143).

| Variable | MI (n = 72) | | Controls (n = 71) | | p-values |
|---|---|---|---|---|---|
| Age, yrs | 64.50 | (55.00-73.50) | 60.00 | (52.00-69.50) | 0.15 |
| Sex (Male), n (%) | 51 | (70.8) | 47 | (66.2) | 0.68 |
| Diabetes mellitus, n (%) | 25 | (34.7) | 20 | (28.2) | 0.51 |
| Hypertension, n (%) | 54 | (75.0) | 42 | (59.2) | 0.07 |
| E, m/s | 0.78 | (0.59-0.99) | 0.76 | (0.63-0.89) | 0.62 |
| A, m/s | 0.75 | (0.61-0.89) | 0.79 | (0.70-0.92) | 0.16 |
| E/A | 0.92 | (0.76-1.16) | 0.87 | (0.74-1.06) | 0.38 |
| Average e′, cm/s | 6.44 | (5.40-7.81) | 8.96 | (7.97-11.30) | <0.0001 |
| E/e′ | 11.57 | (8.51-14.93) | 7.62 | (6.71-9.15) | <0.0001 |
| LVEDV, mL | 106.78 | (82.57-131.55) | 78.36 | (70.13-101.82) | <0.0001 |
| LVESV, mL | 53.63 | (39.31-78.37) | 28.02 | (23.55-36.84) | <0.0001 |
| LVEF, % | 47.49 | (37.84-56.29) | 63.34 | (59.93-67.93) | <0.0001 |
| LAVi, ml/m2 | 21.05 | (16.89-25.86) | 22.92 | (18.09-28.17) | 0.15 |
| LVMi, g/m2 | 85.31 | (74.25-98.86) | 71.23 | (63.05-80.71) | <0.0001 |
| WMSI | 2.08 | (1.27-2.42) | 1.08 | (1.00-1.23) | <0.0001 |
| LS, % | −10.31 | (−13.82-7.75) | −17.30 | (−18.85-15.66) | <0.0001 |

TABLE 6
Clinical characteristics for the cohort from source B (n = 129).

| Variable | MI (n = 38) | | Controls (n = 91) | | p-values |
|---|---|---|---|---|---|
| Age, yrs | 57.00 | (51.25-67.00) | 61.00 | (51.00-68.00) | 0.54 |
| Sex (Male), n (%) | 29 | (76.3) | 54 | (59.3) | 0.10 |
| Diabetes mellitus | 6 | (15.8) | 17 | (18.7) | 0.89 |
| Hypertension, n (%) | 18 | (47.4) | 52 | (57.1) | 0.41 |
| E, m/s | 0.69 | (0.61-0.90) | 0.69 | (0.58-0.84) | 0.41 |
| A, m/s | 0.72 | (0.56-0.80) | 0.69 | (0.55-0.80) | 0.85 |
| E/A | 1.10 | (0.87-1.26) | 1.00 | (0.80-1.30) | 0.41 |
| Average e′, cm/s | 6.60 | (5.32-7.69) | 7.60 | (6.33-8.83) | 0.008 |
| E/e′ | 12.32 | (7.89-16.10) | 8.96 | (7.31-10.84) | 0.007 |
| LVEDV, mL | 95.98 | (87.14-127.51) | 95.95 | (74.22-118.50) | 0.43 |
| LVESV, mL | 45.11 | (34.69-56.18) | 30.30 | (22.90-40.75) | <0.0001 |
| LVEF, % | 49.90 | (40.45-52.83) | 64.00 | (59.00-66.00) | <0.0001 |
| LAVi, ml/m2 | 27.40 | (22.51-33.67) | 32.00 | (27.40-38.42) | 0.01 |
| LVMi, g/m2 | 91.62 | (73.32-98.40) | 76.85 | (66.34-90.34) | 0.08 |
| WMSI | 1.83 | (1.22-2.06) | 1.00 | (1.00-1.00) | <0.0001 |
| LS, % | −12.02 | (−14.29-9.41) | −19.59 | (-22.10-17.49) | <0.0001 |

For continuous variables, the values are mentioned as median (interquartile range), and for categorical variables, the values are mentioned as count (percentage). Continuous variables were compared using a two-sided Student's t-test (if normally distributed per Shapiro-Wilk test) or Mann-Whitney U test (otherwise). Categorical variables were compared using the Chisquared test (if all expected cell counts were ≥5) or Fisher's exact test (otherwise). A=late diastolic transmitral flow velocity; BMI=body mass index; E=early diastolic transmitral flow velocity; e′=early diastolic relaxation velocity at the septal mitral annular position, LS=longitudinal strain; LVEF=left ventricular ejection fraction; LVEDV=left ventricular end-diastolic volume; LVMi=left ventricular mass index; WMSI=wall motion score index; LV=left ventricle

TABLE 7
Clinical characteristics for the cohort from source C (n = 250).

| | | | | | Prospective multivendor database | | | |
|---|---|---|---|---|---|---|---|---|
| | Open-source MIMIC-V-ECHO | | | | (n = 111) | | | |
| | database | | | | Younger | | Younger Controls | |
| | | | Older | | Controls with | | Without risk | |
| | MI | | Controls | | risk factors | | factors | |
| Variable | (n = 32) | | (n = 31) | | (n = 111) | | (n = 76) | |
| Age, yrs | 70.50 | (61.00-79.00) | 82.00 | (72.00-87.00)** | 55.00 | (42.00-66.00) | 32.50 | (24.00-43.00) \# |
| Sex (Male), n | 19 | (59.4) | 21 | (67.7) | 70 | (63.1) | 42 | (55.3) |
| (%) | | | | | | | | |
| Diabetes | 14 | (43.8) | 14 | (45.2) | 25 | (22.5) | 0 | (0.0) \# |
| mellitus, n (%) | | | | | | | | |
| Hypertension, | 10 | (31.2) | 31 | (100.0)* | 75 | (67.6) | 0 | (0.0)* |
| n (%) | | | | | | | | |
| E, m/s | 0.82 | (0.70-0.92) | 0.77 | (0.65-0.87) | 0.77 | (0.67-0.91) | 0.78 | (0.72-0.89) |
| A, m/s | 0.72 | (0.60-0.89) | 1.00 | (0.88-1.16)* | 0.75 | (0.61-0.88) | 0.50 | (0.44-0.64)* |
| E/A | 1.13 | (0.80-1.44) | 0.74 | (0.64-0.89)* | 1.06 | (0.84-1.29) | 1.62 | (1.31-1.80)* |
| Average e′, | 7.38 | (5.59-9.43) | 6.96 | (5.75-8.57) | 9.64 | (7.84-11.83) | 13.23 | (11.66-14.66) \# |
| cm/s | | | | | | | | |
| E/e′ | 9.31 | (8.59-12.45) | 10.69 | (9.11-13.60) | 8.38 | (6.70-9.69) | 6.14 | (5.36-7.07)* |
| LVEDV, mL | 88.33 | (69.52-99.91) | 75.02 | (58.88-99.58) | 94.63 | (76.18-116.35) | 95.38 | (78.46-113.06) |
| LVESV, mL | 36.56 | (25.19-51.68) | 26.15 | (19.24-31.47)* | 34.83 | (27.75-44.13) | 37.88 | (29.61-47.04) |
| LVEF, % | 52.65 | (48.27-65.17) | 69.03 | (64.03-71.90)* | 63.00 | (56.87-68.00) | 61.68 | (53.46-66.00) |
| LAVi, ml/m² | 23.92 | (20.32-30.82) | 28.27 | (20.61-33.78) | 21.62 | (17.85-24.86) | 16.29 | (14.30-20.37)* |
| LVMi, g/m² | 86.29 | (76.66-107.99) | 76.02 | (62.55-92.30) | 65.83 | (55.57-79.74) | 61.59 | (53.16-70.53)* |
| WMSI | 1.00 | (1.00-1.42) | 1.00 | (1.00-1.00)* | 1.00 | (1.00-1.00) | 1.00 | (1.00-1.00)* |
| LS, % | −14.76 | (−17.05-11.95) | −17.84 | (−19.40-16.34)* | −21.03 | (−23.97-18.86) | −22.67 | (−25.11-20.47) \# |

### Prediction of MI Using HCR Features

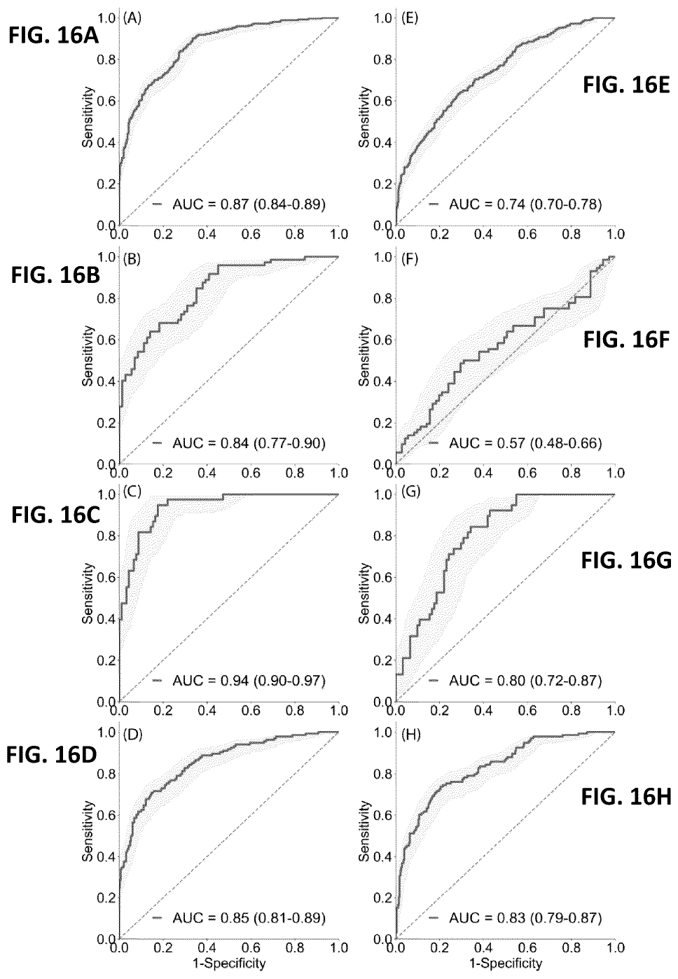

The gradient boost model (XGBoost) emerged as a model for MI prediction, with hyperparameters listed in Table 8. HCR features extracted from frames across the entire cardiac cycle yielded an overall sensitivity of 90.1% (95% CI: 86.4-93.7%), specificity of 66.4% (95% CI: 62.370.7%), F1-score of 71.8% (95% CI: 67.9-75.7%), accuracy of 74.9% (95% CI: 71.978.1%), and AUC of 0.87 (95% CI: 0.84-0.89), based on leave-one-source-out crossvalidation across all the three held-out sets (Table 7). Independent data source-wise performance metrics are presented in Table 7. FIGS. 16A-D presents the independent ROC curves for the three data sources, along with the overall ROC curve representing the comprehensive performance of the model.

TABLE 8
The hyperparameters of the patient-level XGBoost model.

| Parameter | Value |
|---|---|
| Number of trees | 50 |
| Maximum depth | 6 |
| Minimum child weight | 1 |
| Learning rate | 0.6 |
| eta | 0.3 |
| Sample rate | 1 |
| Subsample | 1 |
| Maximum bins | 256 |

TABLE 9
Performance of machine learning models developed using a leave-one-
source-out cross-validation approach with handcrafted radiomics
(HCR) and deep transfer learning (DTL) features.

| | | Accur- | | Sensitiv- | Specif- | F1- | |
|---|---|---|---|---|---|---|---|
| Modal- | | acy | Brier | ity | icity | Score | |
| ity | Sources | (%) | score | (%) | (%) | (%) | AUC |
| HCR | Source | 75.5 | 0.17 | 91.7 (84.3- | 59.2 | 79.0 | 0.84 |
| | A | (67.8- | (0.14- | 97.2) | (48.0- | (71.7- | (0.78- |
| | | 83.2) | 0.20) | | 71.1) | 85.4) | 0.90) |
| | Source | 86.0 | 0.09 | 94.7 (86.1- | 82.4 | 80.0 | 0.94 |
| | B | (80.6- | (0.06- | 100.0) | (74.4- | (71.1- | (0.90- |
| | | 92.2) | 0.12) | | 90.4) | 88.6) | 0.97) |
| | Source | 80.6 | 0.17 | 70.7 (63.4- | 85.3 | 70.1 | 0.85 |
| | C | (76.7- | (0.14- | 77.9) | (81.3- | (63.7- | (0.82- |
| | | 84.5) | 0.19) | | 89.3) | 76.2) | 0.89) |
| | Overall | 74.9 | 0.15 | 90.1 (86.4- | 66.4 | 71.8 | 0.87 |
| | | (71.9- | (0.14- | 93.7) | (62.3- | (67.9- | (0.84- |
| | | 78.1) | 0.17) | | 70.7) | 75.7) | 0.89) |
| DTL | Source | 59.4 | 0.29 | 48.6 (37.3- | 70.4 | 54.7 | 0.57 |
| | A | (51.7- | (0.25- | 60.6) | (59.7- | (44.4- | (0.48- |
| | | 67.8) | 0.34) | | 80.3) | 64.3) | 0.67) |
| | Source | 71.3 | 0.22 | 84.2 (71.1- | 65.9 | 63.4 | 0.80 |
| | B | (63.6- | (0.16- | 94.6) | (56.3- | (51.1- | (0.72- |
| | | 78.3) | 0.29) | | 75.5) | 72.7) | 0.87) |
| | Source | 77.7 | 0.23 | 73.7 (66.4- | 79.6 | 68.1 | 0.83 |
| | C | (73.5- | (0.20- | 80.8) | (75.2- | (61.7- | (0.79- |
| | | 81.6) | 0.27) | | 84.2) | 73.8) | 0.87) |
| | Overall | 68.6 | 0.24 | 63.4 (57.2- | 71.4 | 58.9 | 0.74 |
| | | (65.3- | (0.22- | 69.4) | (67.3- | (54.0- | (0.70- |
| | | 71.9) | 0.27) | | 75.6) | 63.9) | 0.77) |

Data in parentheses are 95% confidence intervals computed using the bootstrapping technique.
HCR = handcrafted radiomics,
DTL = deep-learning-based radiomics,
AUC = area-under-the-curve,
HCR = hand-crafted radiomics, and
DTL = deep transfer learning

### Prediction of MI Using DTL Features

Similarly, among the four 3DCNN models, the slow-fast ResNet50-based model provided better performance among the four 3D-CNN models (Table 10). The ML model with DTL features extracted from this 3DCNN yielded an overall sensitivity of 63.4% (95% CI: 57.2-69.4%), specificity of 71.4% (95% CI: 67.3-75.6%), F1-score of 58.9% (95% CI: 54.0-63.9%), the accuracy of 68.6% (95% CI: 65.3-71.9%), and AUC of 0.74 (95% CI: 0.70-0.77) (Table 4). FIGS. 16E-H present the independent ROC curves for the three sources, along with the overall ROC curve representing the comprehensive performance of the DTL-based ML model.

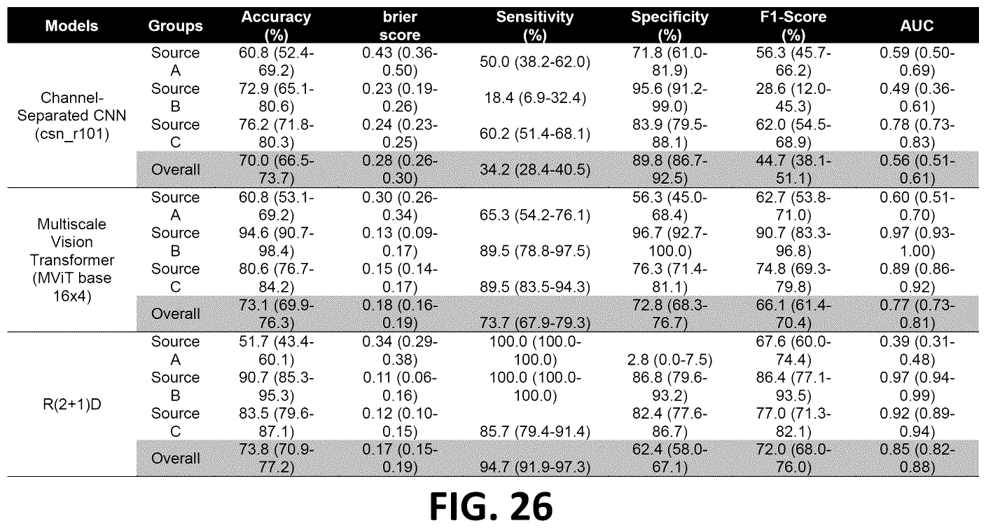

Table 10 (FIG. 26) Performance of ML models on the training dataset with cross-validation and test dataset, utilizing DTL features from cardiac cycle extracted from four 3D-CNN architectures.

### Model Prediction Adjusted for Echocardiography Variables

Significant univariate echocardiographic predictors of MI in the entire cohort are presented in Table 8. ML prediction probability was concatenated from each independent held-out set to obtain the results for the whole population and compared with other echocardiographic measurements. LV mass index, end-systolic volume, ejection fraction, ratio of early diastolic mitral inflow velocity to early diastolic mitral annulus velocity, wall motion score index, average longitudinal strain, and ML probability were significant univariate predictors of MI(p<0.05). However, on multivariable regression, average longitudinal strain [adjusted odds ratio, 1.52 [1.271.82], p<0.0001] and ML probability [adjusted odds ratio, 1.03 [1.01-1.05], p<0.0001] were independent predictors of MI.

TABLE 11
Univariate and multivariate analysis for predicting myocardial infarction
using echocardiographic variables.

| Variable | OR (95% CI) | p-value | OR (95% CI) | p-value |
|---|---|---|---|---|
| LVMi, g/m² | 1.03 (1.02-1.04) | <0.0001 | 1.01 (0.99-1.04) | 0.3648 |
| LVESV, mL | 1.05 (1.04-1.06) | <0.0001 | 0.99 (0.96-1.03) | 0.6276 |
| LVEF, % | 0.88 (0.85-0.90) | <0.0001 | 0.94 (0.88-1.00) | 0.0594 |
| E/A ratio | 0.86 (0.52-1.41) | 0.5376 | — | — |
| Average E/e′ | 1.21 (1.14-1.28) | <0.0001 | 1.11 (0.98-1.25) | 0.1041 |
| WMSI | 31.91 (15.82-64.36) | <0.0001 | 0.48 (0.10-2.23) | 0.3489 |
| Averaged LS, % | 1.71 (1.54-1.89) | <0.0001 | 1.52 (1.27-1.82) | <0.0001 |
| ML | 1.05 (1.04-1.06) | <0.0001 | 1.03 (1.01-1.05) | <0.0001 |
| probability, % | | | | |

A = late diastolic transmitral flow velocity;
E = early diastolic transmitral flow velocity;
e′ = early diastolic relaxation velocity at the septal mitral annular position;
LVEF = left ventricular ejection fraction;
LVMi = left ventricular mass index;
LV = left ventricle;
LS = longitudinal strain;
WMSI = wall motion score index

### Incremental Value of the Ultrasound Radiomics ML Model

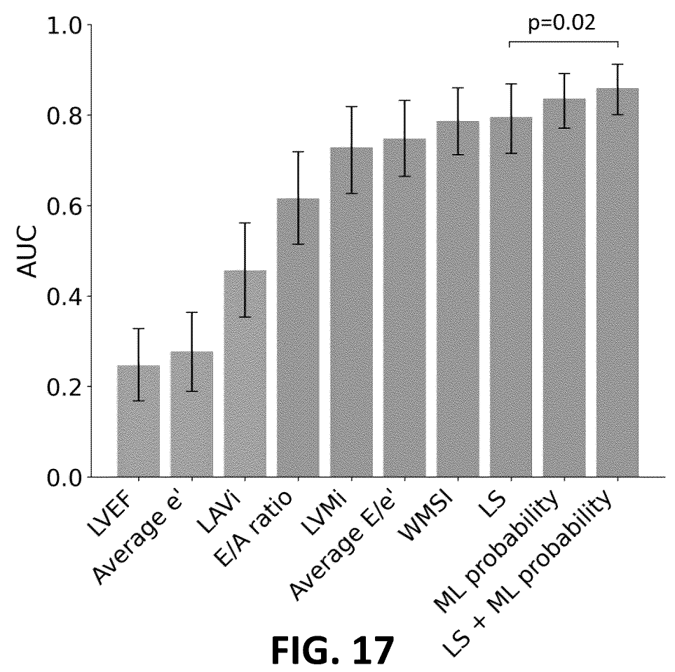

To explore whether the ML model could help discriminate the presence or absence of MI in patients with an abnormal echocardiogram, the incremental value of the ML model for patients with myocardial dysfunction (LS<16%) was assessed. ML showed an AUC of 0.84(95% CI: 0.77-0.89). A comparison with other conventional echocardiographic parameters is shown in FIG. 17. Besides outperforming other echocardiographic biomarkers, a combination of GLS and ML probability further improved the prediction over GLS alone [AUC of 0.86 (95% CI: 0.80-0.91) vs. 0.80 (95% CI: 0.72-0.87), p=0.02].

### Ultrasound Radiomics Signatures of Infarcted Myocardium

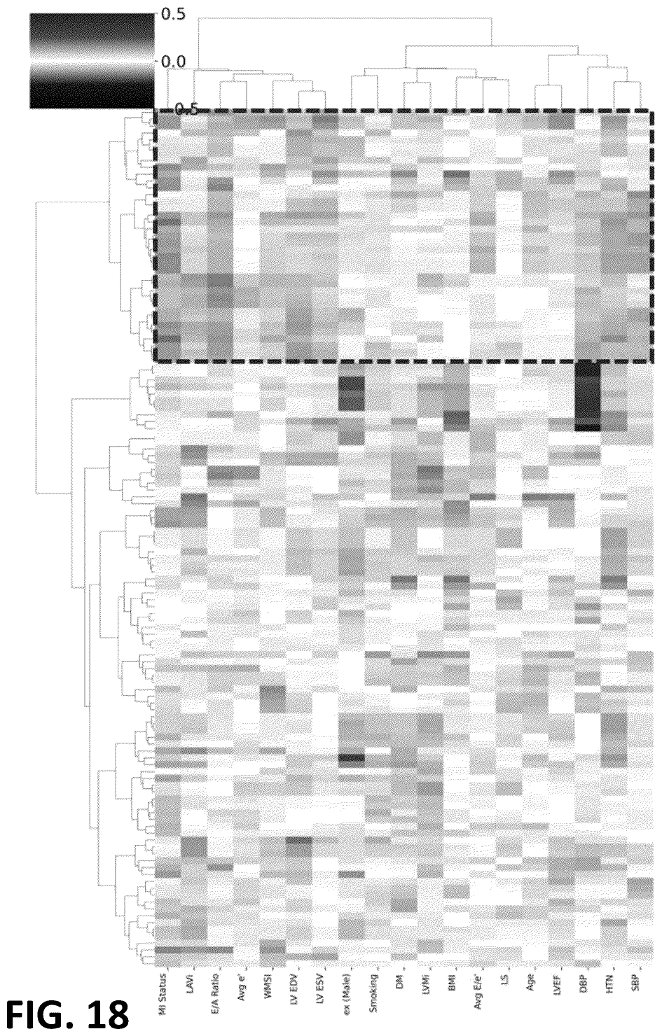

To understand whether the ultrasound radiomic features that distinguish infarcted myocardium are unique from those that are associated with age-, sex-, and risk factor-related changes, an exploratory analysis was performed using agglomerative hierarchical clustering (FIG. 18) on data from Data Source C (n=250). Clustering revealed distinct ultrasonographic patterns: MI cases exhibited unique ultrasound radiomic signatures which were clustered separately from those related to age and risk factors (e.g., diabetes, hypertension). Analyzing the feature of importance for the XGboost model developed using LOSO-CV revealed that eight of the top 20 features identified in the feature of importance analysis belong to the highlighted cluster shown in FIG. 18 and were unique for identifying MI for non-MI controls.

### Parametric Visualization of Infarcted Myocardium

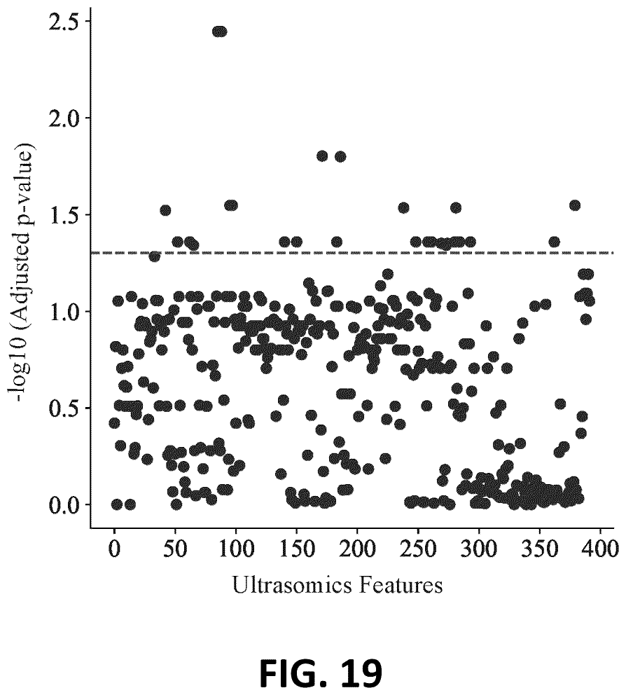

CMR segmental hyperenhancement data from the 40 STEMI patients were used to distinguish infarcted and non-infarcted segments. Ultrasound radiomic features averaged for infarcted and non-infarcted segments in each individual are presented in a Manhattan plot (FIG. 19). Among a series of texture features identified, the features were iteratively displayed on the cardiac ultrasound images. The gray-level nonuniformity feature extracted from the gray-level dependence matrix was identified. This feature discriminated between infarcted and non-infarcted segments (p=0.0007, Table 12) and enabled differentiation of patients with and without MI (FIGS. 20A-F).

TABLE 12
A pairwise comparison to identify the features that discriminate the
infarcted segments against the non-infarcted segments within a view.

| Ultrasound Radiomics Features | p-value |
|---|---|
| NGTDM Strength at the ED frame | p <0.0001 |
| NGTDM Coarseness at ED frame | p <0.0001 |
| GLSZM Large Area High Gray Level Emphasis at ES frame | 0.00012 |
| NGTDM Strength at ES frame | 0.0002 |
| NGTDM Coarseness from spectral variations | 0.0005 |
| GLDM Gray Level Non-Uniformity from Temporal Variations | 0.0007 |
| NGTDM Coarseness from temporal variations | 0.0007 |
| GLDM Gray Level Non-Uniformity from ED frame | 0.0007 |

NGTDM: Neighbouring Gray Tone Difference Matrix, GLSZM: Gray Level Size Zone Matrix,
GLDM: Gray Level Dependence Matrix

### Harmonization and Reproducibility Analysis

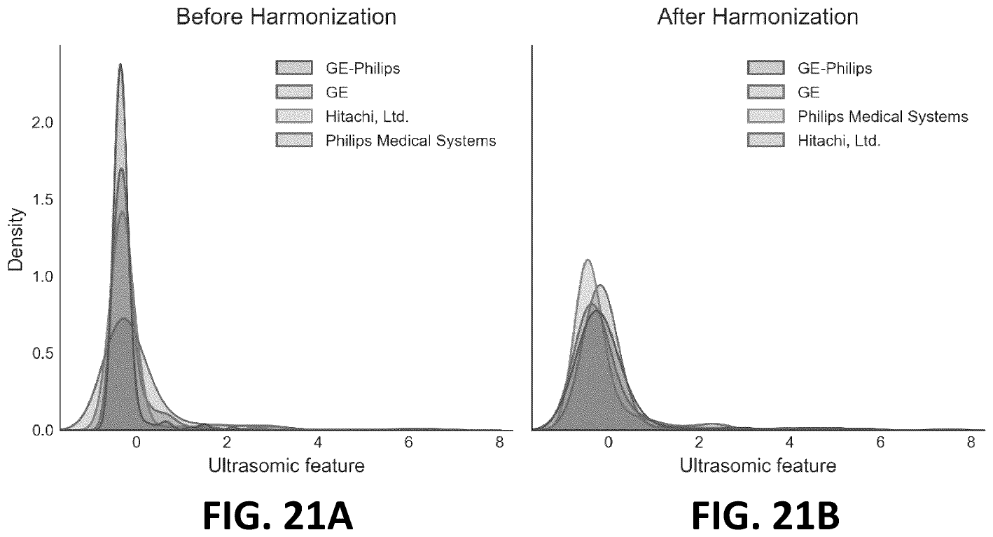

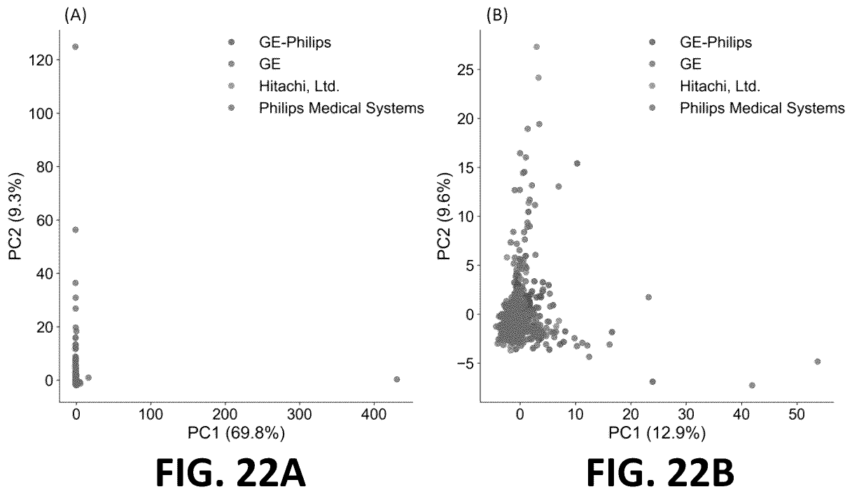

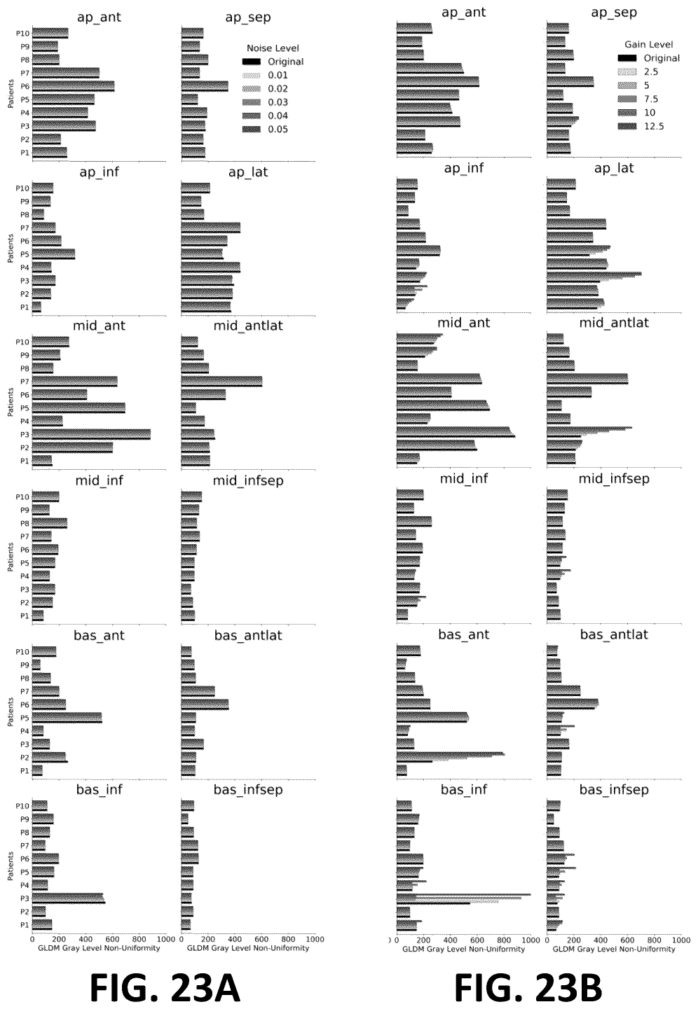

The effect of ComBat harmonization in reducing vendor-specific biases in ultrasound radiomic feature distribution is shown in FIG. 21A-B and FIG. 22A-B. This correction enhanced model performance substantially, with the AUC increasing from 0.74 (95% CI: 0.70-0.78) to 0.87 (95% CI: 0.84-0.89). In previous studies, the stability of ultrasound radiomics markers despite image quality variability was confirmed. Similarly, in the present study, apical views were analyzed (FIGS. 23A-B, 24A-B) and the impact of gain and Gaussian noise was tested. Ultrasound radiomics features from static and dynamic image loops showed high consistency, with an ICC of 0.98 (p<0.0001). The feasibility analysis for the ML model demonstrated stability under noise, with minimal AUC variation (<5%) (Table 13).

TABLE 13
A pairwise comparison to identify the features that discriminate the
infarcted segments against the non-infarcted segments within a view.

| Models for | Training | % variations in | Test | % variations |
|---|---|---|---|---|
| Feasibility Analysis | Performance | training AUC | Performance | in test AUC |
| Baseline Model | 0.89 | NA | 0.88 | NA |
| Model with 0.01 | 0.88 | 1.1 | 0.86 | 2.3 |
| noise | | | | |
| Model with 0.03 | 0.84 | 4.5 | 0.85 | 1.2 |
| noise | | | | |
| Model with 0.05 | 0.85 | 1.2 | 0.83 | 2.4 |
| noise | | | | |
| Average | 0.86 + 0.02 | 2.3 ± 2.0 | 0.85 ± 0.02 | 1.9 ± 0.7 |
| performance | | | | |

Radiomics features extracted from cardiac ultrasound images can vary significantly due to differences in imaging protocols and scanner settings across centers. This study acquired imaging data from multiple centers using various ultrasound systems, including GE Vingmed, Philips Medical Systems, and Hitachi. To address scanner-related variability and reduce batch effects in the radiomics data, the ComBat harmonization technique was applied-a widely used and robust method for correcting batch effects. ComBat was chosen for its ability to adjust both location (mean) and scale (variance) of feature distributions, making it particularly suitable for harmonizing radiomics data.

A source-wise harmonization approach was followed, where HCR and DTL features from each source were independently harmonized using a separate multivendor reference dataset not used for model training or evaluation (n=801); it served solely for harmonization. Radiomics features were available for all patients from either A2C or A4C views, with both views present in 73% of cases. For the rest, features from one view were sufficient. Median imputation was applied for missing values during ComBat harmonization. By aligning each source to this standard reference, ComBat effectively corrected scanner-induced variability, helping ensure consistency and comparability of features across different sources.

FIGS. 22A-22B illustrate the effect of ComBat harmonization on vendor-related variability in ultrasound radiomics features. (A) PCA plots of all radiomics features, colored by vendor, demonstrate that prior to harmonization, features are strongly clustered by vendor, indicating significant batch effects. After ComBat harmonization (B), vendor-related separation is substantially reduced, and features from different vendors align more closely in the feature space. These results confirm that ComBat effectively mitigates scanner- and protocol-induced biases, allowing for improved generalizability in downstream analyses.

FIGS. 23A-23B illustrate the impact of (A) Gaussian noise perturbations and (B) gain-level variations in image quality on ultrasound radiomics features derived from static segmental images.

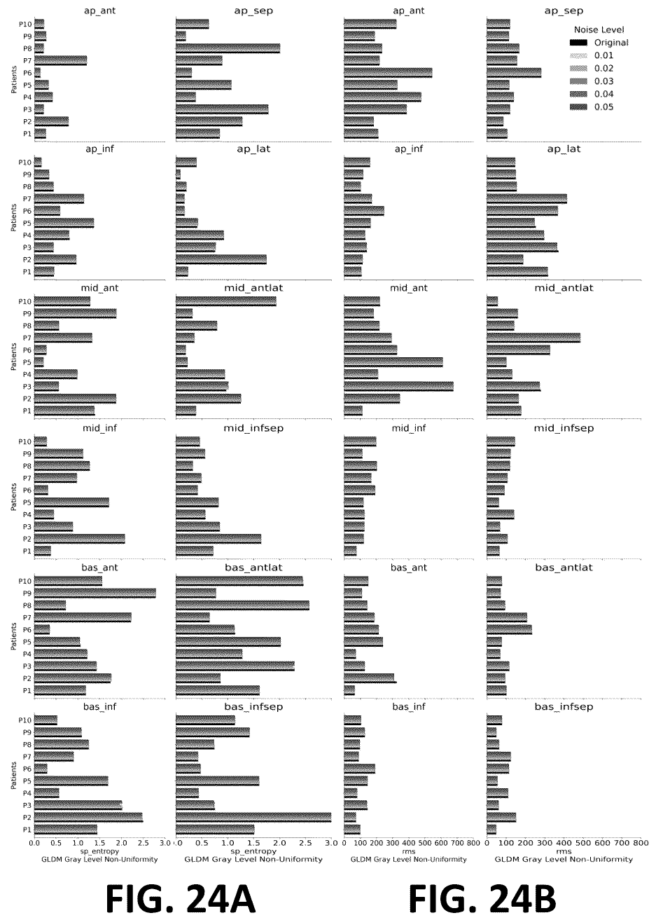

FIGS. 24A-24B illustrate the effect of Gaussian noise perturbations in image quality on (A) temporal and (B) spectral variations in ultrasound radiomics features derived from segmental images.

### DISCUSSION

The structural components of the myocardium affect its acoustic properties. Infarcted myocardium shows stretched and rearranged necrotic myocytes, surrounded by contracted myocytes, edema, hemorrhage, and inflammatory repair processes. These changes within the myocardium influence ultrasound signal intensity distributions. The described technology provides computational approaches that support radiomics applications for broader adoption in clinical practice. For example, the described technology supports use of ultrasound radiomics features even from apical echocardiography views for developing ML models.

Convolutional Neural Networks (CNNs) are effective for medical image analysis and have shown promising results with transfer learning from large ImageNet-trained models. Static and dynamic features were explored, incorporating cardiac cycle variations using radiomics and 3D-CNN. Dynamic models generally performed better than static models, but handcrafted ultrasound radiomics outperformed 3D-CNN-based deep features in myocardial infarction prediction. Handcrafted ultrasound radiomics, thus, may offer an advantage in capturing intricate tissue-level changes with smaller sample sizes than deep features extracted by 3D-CNNs, which typically require larger datasets due to their data-hungry nature.

Amongst various clinical variables indicating prognostic significance in myocardial infarction, longitudinal strain, measured using speckle tracking echocardiography, is considered the most sensitive marker for identifying LV dysfunction. Deep learning-based techniques may reduce interobserver variability in assessing longitudinal strain, regional wall motion score index, and LV ejection fraction. therefore, a previously validated deep learning platform was used in this study (US2ai.com²²) to measure these clinical parameters and their interaction with the ML model for predicting MI. Notably, logistic regression analysis showed the ability of the described technology to independently predict MI even after adjusting for average longitudinal strain and other echocardiographic parameters (Table 5). Ultrasound radiomics provided significant incremental value over the averaged longitudinal strain in distinguishing MI, (FIG. 17). Since regional or global LV dysfunction can arise from various causes, identifying MI as the underlying factor holds clinical significance for patients presenting with acute coronary syndrome.

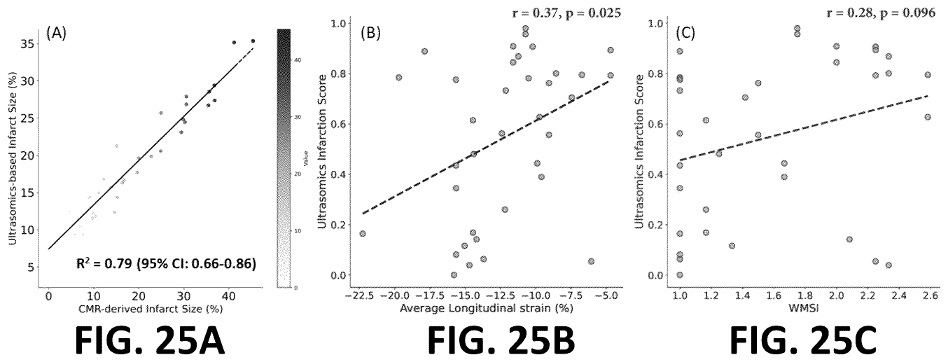

Some applications of the disclosed technology may support estimating total infarct size. The system can quantify infarct size by analyzing the spatial distribution of radiomic features across myocardial segments. This quantification provides clinicians with precise measurements of affected tissue volume, enabling more accurate risk stratification. The parametric visualization capabilities allow for clear delineation between infarcted and viable myocardium, enhancing diagnostic confidence. TA predictive analysis was performed using segmental ultrasound radiomics features from both a2c and a4c views, selecting eight key features through recursive feature elimination. The resulting regression model strongly predicted CMR-derived infarct size, showing a robust association with an R² of 0.79 (95% CI: 0.66-0.86, FIGS. 24A-B). The correlation between ML probability and longitudinal strain was 0.37 (p=0.025), and with WMSI, it was 0.28 (p=0.096). While these results are promising, further validation is needed and has been acknowledged in the limitation section. FIGS. 25A-C illustrate (A) prediction of infarct size based on ultrasound radiomics and its comparison with CMR-derived infarct size, (B) association between ultrasound radiomics score and average longitudinal strain, and (C) association with WMSI.

In some implementations, the described technology may be integrated directly with existing ultrasound systems, offering automated cardiac image analysis. Following the image identification steps, the software automatically performs segmentation, quality control, and radiomics feature extraction at the present stage, requiring approximately 3-5 minutes per patient. Results can be displayed as visual overlays, ensuring transparency for straightforward interpretation.

Accordingly, this example experiment provides various examples of aspects of the described technology, such as the use of multicenter and opensource data to develop machine-learning models, validation using core-laboratory assessed multi-institutional clinical trial database using cardiac magnetic resonance imaging as a gold standard for infarct quantification, comparison with conventional echocardiography features, the comparison with transfer-learning derived deep features and 3D CNN models, the use of harmonization techniques across multivendor echocardiography settings and the use of leave-source-out cross validation for external validation.

### Example Implementations

The ultrasound radiomics system 1400 may comprise at least one computing device 900. For example, as shown in FIG. 9, the computing device 900 may include a processor 903, a memory 906, and a data store 912. The memory 906 may store a myocardial ultrasonic fingerprinting application 915 that, when executed by the processor 903, causes the computing device 900 to perform various operations related to myocardial infarction detection and analysis.

The myocardial ultrasonic fingerprinting application 915 may extract a plurality of radiomic features from an ultrasound scan associated with a person. For instance, as illustrated in FIG. 14, the data extraction 1401 component of the ultrasound radiomics system 1400 may process DICOM images 1402, which may include an apical two chamber view 1403 and an apical four chamber view 1404. These views may be used to generate a segmented image 1405 and a segmented image 1406, which are further divided into segments 1407 and segments 1408.

For example, a feature identification 1409 component may analyze the segmented images to extract multiple types of features, including a shape feature 1410, a first order feature 1411, and a texture feature 1412. These extracted features may comprise both dynamic features and static features. Dynamic features may capture temporal changes across the cardiac cycle, while static features may represent fixed characteristics of the myocardium. The extracted features may be particularly useful for identifying various types of cardiac muscle injury or damage resulting from diverse causes.

The myocardial ultrasonic fingerprinting application 915 may identify one or more myocardial textures by applying the extracted plurality of radiomic features to at least one phenotyping model. For instance, as shown in FIG. 14, this process may be implemented through the model development 1413 component, which includes a training protocol 1414 and a classifier network 1415. The phenotyping models may be trained to detect various cardiac injuries, including myocardial infarction, which results from insufficient blood flow to cardiac tissue. In addition, the phenotyping models may detect other cardiac injuries such as myocarditis caused by viral, bacterial, or fungal infections; cardiac muscle damage from inflammatory conditions like sarcoidosis or autoimmune disorders; drug-induced cardiotoxicity from chemotherapeutic agents such as anthracyclines, trastuzumab, or tyrosine kinase inhibitors; alcohol-induced cardiomyopathy; stress-induced cardiomyopathy (Takotsubo syndrome); and radiation-induced cardiac injury. In some cases, the phenotyping models may be specifically trained to differentiate between acute and chronic cardiac injuries based on the temporal evolution of radiomic features.

The application may compare the one or more myocardial textures to at least one phenotype cluster for at least one known condition. For example, this comparison may be visualized using patient cluster mappings 203, such as the circular network cluster 203a or linear network cluster 203b shown in FIGS. 2A-2B. As another example, as illustrated in FIG. 6, the identification of a cardiac injury may be based at least in part on matching the radiomic features to a gradient of the patient cluster 609. For instance, in cases of myocardial infarction, the radiomic features may exhibit specific patterns related to tissue necrosis and subsequent fibrosis, while in cases of myocarditis, the features may reflect diffuse inflammatory changes with edema and cellular infiltration. Cardiotoxicity from chemotherapeutic agents may present with distinct radiomic signatures that evolve overtime, with early changes reflecting edema and later changes indicating fibrosis. In some cases, the system may detect subclinical cardiac injury before conventional echocardiographic parameters show abnormalities, allowing for earlier intervention. The phenotype clusters may be organized to reflect not only the type of cardiac injury but also the severity and chronicity, providing clinically relevant stratification for patient management.

In some implementations, the myocardial ultrasonic fingerprinting application 915 may select a portion of the plurality of radiomics features. The application may then select at least one of the one or more phenotyping models based at least in part on this portion of radiomics features. The determination of myocardial infarction may be based at least in part on the selected portion of radiomics features and the chosen phenotyping model.

The myocardial ultrasonic fingerprinting application 915 may quantify a size of an infarct associated with the myocardial infarction. For example, as shown in FIGS. 5A and 5B, this quantification may be based on the analysis of myocardial fibrosis textures 503. The application may also locate infarcted myocardium based at least in part on the one or more myocardial characteristics identified through the radiomic analysis.

In some cases, the myocardial ultrasonic fingerprinting application 915 may create a parametric map of the infarcted myocardium. This parametric map may be based on a paired cardiac magnetic resonance (CMR) assessment. The creation of such a map may involve correlating the radiomic features extracted from ultrasound images with CMR-derived data to provide a comprehensive visualization of the infarcted region.

The ultrasound scan used for extracting radiomic features may comprise an apical view ultrasound scan. In some implementations, the medical images used to determine the myocardial infarction may consist solely of one or more ultrasound scans, demonstrating the potential of the described technology to provide valuable diagnostic information without requiring additional imaging modalities.

For example, the performance of the myocardial infarction detection and analysis may be evaluated using the performance graph 1416 component of the ultrasound radiomics system 1400. This graph may display various metrics such as sensitivity, specificity, and area under the receiver operating characteristic curve, providing a comprehensive assessment of the system's diagnostic capabilities.

In myocardial infarction detection, a localized infarct may lead to global changes in the heart's geometry—a fundamentally distributed phenomenon. As a result, when estimating local effects in a specific region of the myocardium, tissue structure and motion alterations may manifest throughout the heart. Consequently, pixel-level statistical features that reflect these changes may be extracted from multiple imaging views, including those that do not directly capture the infarcted region. For instance, as shown in FIG. 20A-20F, different imaging views show parametric visualization of infarcted myocardium using both standard grayscale ultrasound and enhanced visualization methods with color overlays. Global cardiac properties-such as changes in left ventricular (LV) mass, remodeling, and ejection fraction—may be inferred from pixel-level data across one or more views. Localized pathology, like myocardial infarction, may induce widespread alterations in cardiac morphology and function. Due to the heart's integrated biomechanical and functional response, these global effects may be detected through pixel-level statistical patterns, even in views that do not include the infarct itself.

For example, as shown in FIG. 5A-5C, the myocardial ultrasonic fingerprinting application 815 may identify negative or positive myocardial fibrosis textures 503 through radiomic-based clustering of static cardiac ultrasound images 512. The ultrasound radiomics system 1400 may estimate the local damage, such as the infarct size, using the global-to-local associations extracted through the feature identification component 1409. In some cases, the ultrasound radiomics system 1400 may utilize both apical two chamber view 1403 and apical four chamber view 1404 to estimate infarct size. For instance, the ultrasound radiomics system 1400 may extract shape feature 1410, first order feature 1411, and texture feature 1412 from both views and apply a regression model to predict infarct size. In one implementation, the system may select features through recursive feature elimination (e.g., 6-10 features, with 8 features as a particular example) from both views and generate a predictive model. This approach may enable quantification of infarct size as a percentage of total myocardial volume without requiring contrast agents or additional imaging modalities. For example, as illustrated in FIG. 19, the Manhattan plot visualization demonstrates how different ultrasonic features may show varying levels of statistical significance, with features above the significance threshold potentially representing those that significantly differentiate between infarcted and non-infarcted segments. This approach may be extended to other disease states that impact locally, like scars, infiltrative diseases like amyloid, and other conditions-essentially, the ultrasound radiomics system 1400 may estimate the amount of disease-impacted myocardium. As another example, the hierarchical clustering heatmap shown in FIG. 18 further demonstrates how distinct patterns in the data may reveal structured relationships between different features or samples being analyzed, with clear groupings of similar values indicated by consistent coloring within certain regions. Thus, the ultrasound radiomics system 1400 may estimate both global and local changes, and each has diagnostic and prognostic implications. For instance, the texture-based phenotyping 300 shown in FIG. 3 may further enhance the ability to identify quantitative features of myocardial tissue with more predictive accuracy than conventional methods, allowing for identification of pathological cardiovascular changes earlier than traditional qualitative imaging.

Through the combination of advanced image processing techniques, machine learning algorithms, and clinical domain knowledge, the described ultrasound radiomics system 1400 and associated myocardial ultrasonic fingerprinting application 915 may provide a powerful tool for the detection, localization, and characterization of myocardial infarction using readily available ultrasound imaging technology.

In addition to the foregoing, the various embodiments of the present disclosure include, but are not limited to, the embodiments set forth in the following clauses.

Clause 1. A system, comprising: at least one computing device; and at least one application executable on the at least one computing device, wherein, when executed, the at least one application causes the at least one computing device to at least: extract a plurality of radiomic features from an ultrasound scan associated with a patient; determine one or more myocardial characteristics by applying the extracted plurality of radiomic features to one or more phenotyping models; and identify a cardiac injury associated with the patient based at least in part on the one or more myocardial characteristics and matching the extracted plurality of radiomic features to a patient cluster.

Clause 2. The system of clause 1, wherein the cardiac injury is a myocardial infarction, and wherein, when executed, the at least one application further causes the at least one computing device to at least quantify a size of an infarct associated with the myocardial infarction.

Clause 3. The system of clause 1, wherein the cardiac injury is a myocardial infarction, and wherein, when executed, the at least one application further causes the at least one computing device to at least locate infarcted myocardium based at least in part on the one or more myocardial characteristics.

Clause 4. The system of clause 3, wherein, when executed, the at least one application further causes the at least one computing device to at least create a parametric map of the infarcted myocardium.

Clause 5. The system of clause 4, wherein, when executed, the at least one application further causes the at least one computing device to at least create a parametric map based on a paired cardiac magnetic resonance (CMR) assessment.

Clause 6. The system of any one of clauses 1-5, wherein, when executed, the at least one application further causes the at least one computing device to extract the plurality of radiomic features from a plurality of ultrasound scans associated with the patient, the plurality of ultrasound scans comprising a plurality of views.

Clause 7. The system of any one of clauses 1-6, wherein the plurality of radiomic features comprise dynamic features and static features.

Clause 8. The system of any one of clauses 1-7, wherein, when executed, the at least one application further causes the at least one computing device to at least identify one or more myocardial textures based at least in part on a clustering of the extracted plurality of radiomic features.

Clause 9. The system of any one of clauses 1-8, wherein the cardiac injury is a myocardial infarction, and wherein the identification of the myocardial infarction is further based at least in part on matching the radiomic features to a gradient of the patient cluster.

Clause 10. The system of any one of clauses 1-9, wherein, when executed, the at least one application further causes the at least one computing device to at least determine a global-to-local association and estimate local cardiac damage based at least in part on the global-to-local association.

Clause 11. The system of any one of clauses 1-10, wherein, when executed, the at least one application further causes the at least one computing device to at least estimate global cardiac damage based at least in part on the extracted plurality of radiomic features.

Clause 12. A method, comprising: extracting, via at least one computing device, a plurality of radiomic features from an ultrasound scan associated with a person; identifying, via the at least one computing device, one or more myocardial textures by applying the extracted plurality of radiomic features to at least one phenotyping model; comparing, via the at least one computing device, the one or more myocardial textures to at least one phenotype cluster for at least one known condition; and determining, via the at least one computing device, a cardiac injury associated with the person based at least in part on the one or more myocardial textures being matched with one or more of the at least one phenotype cluster, and the extracted plurality of radiomic features.

Clause 13. The method of clause 12, wherein the cardiac injury is a myocardial infarction, and further comprising quantifying, via the at least one computing device, a size of an infarct associated with the myocardial infarction based at least in part on the one or more myocardial textures.

Clause 14. The method of clause 12, wherein medical images used to determine the cardiac injury consist of one or more ultrasound scans.

Clause 15. The method of clause 12, wherein the cardiac injury is a myocardial infarction, and further comprising locating, via the at least one computing device, an infarct associated with the myocardial infarction based at least in part on the one or more myocardial textures.

Clause 16. The method of clause 15, further comprising creating, via the at least one computing device, a parametric map of an infarcted myocardium.

Clause 17. The method of clause 16, further comprising creating, via the at least one computing device, the parametric map based on a paired cardiac magnetic resonance (CMR) assessment.

Clause 18. The method of any one of clauses 12-17, wherein the cardiac injury is a myocardial infarction, and wherein the determination of the myocardial infarction is further based at least in part on matching the radiomic features to a gradient of the at least one phenotype cluster.

Clause 19. The method of any one of clauses 12-18, further comprising: selecting, via the at least one computing device, a portion of the plurality of radiomics features; selecting, via the at least one computing device, the at least one phenotyping model based at least in part on the portion of the plurality of radiomics features; and determining the cardiac injury based at least in part on the portion of the plurality of radiomics features and the at least one phenotyping model.

Clause 20. The method of any one of clauses 12-19, wherein the ultrasound scan comprises an apical view ultrasound scan and the plurality of features comprise static features and dynamic features.

Clause 21. The method of any one of clauses 12-20, wherein extracting the plurality of radiomic features from the ultrasound scan further comprises detecting pixel-based patterns in the ultrasound scan.

Clause 22. The method of any one of clauses 12-21, further comprising identifying, via the at least one computing device, at least one selected region of interest in the ultrasound scan, wherein the plurality of radiomic features are extracted from the at least one selected region of interest in the ultrasound scan.

Clause 23. The method of any one of clauses 12-22, wherein the one or more phenotyping models comprise at least one of a neural network classifier, a support vector machine (SVM) classifier, or a deep learning classifier.

Clause 24. The method of any one of clauses 12-23, wherein the ultrasound scan comprises a static two-dimensional cardiac ultrasound image.

Clause 25. The method of any one of clauses 12-24, further comprising obtaining, via at least one computing device, the ultrasound scan from an ultrasound capturing device in data communication with the at least one computing device.

Clause 26. A system for identifying myocardial infarction, comprising: at least one computing device; and at least one application executable on the at least one computing device, wherein, when executed, the at least one application causes the at least one computing device to at least: extract a plurality of radiomic features from an ultrasound scan associated with a patient; determine one or more myocardial characteristics by applying the extracted plurality of radiomic features to one or more phenotyping models; and identify a myocardial infarction associated with the patient based at least in part on the one or more myocardial characteristics and matching the extracted plurality of radiomic features to a patient cluster.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

What is claimed is:

1. A system, comprising:
at least one computing device; and
at least one application executable on the at least one computing device, wherein, when executed, the at least one application causes the at least one computing device to at least:
extract a plurality of radiomic features from an ultrasound scan associated with a patient;
determine one or more myocardial characteristics by applying the extracted plurality of radiomic features to one or more phenotyping models; and
identify a cardiac injury associated with the patient based at least in part on the one or more myocardial characteristics and matching the extracted plurality of radiomic features to a patient cluster.

2. The system of claim 1, wherein the cardiac injury is a myocardial infarction, and wherein, when executed, the at least one application further causes the at least one computing device to at least quantify a size of an infarct associated with the myocardial infarction.

3. The system of claim 1, wherein the cardiac injury is a myocardial infarction, and wherein, when executed, the at least one application further causes the at least one computing device to at least locate infarcted myocardium based at least in part on the one or more myocardial characteristics.

4. The system of claim 3, wherein, when executed, the at least one application further causes the at least one computing device to at least create a parametric map of the infarcted myocardium.

5. The system of claim 4, wherein, when executed, the at least one application further causes the at least one computing device to at least create a parametric map based on a paired cardia magnetic resonance (CMR) assessment.

6. The system of claim 1, wherein, when executed, the at least one application further causes the at least one computing device to extract the plurality of radiomic features from a plurality of ultrasound scans associated with the patient, the plurality of ultrasound scans comprising a plurality of views.

7. The system of claim 1, wherein the plurality of radiomic features comprise dynamic features and static features.

8. The system of claim 1, wherein, when executed, the at least one application further causes the at least one computing device to at least identify one or more myocardial textures based at least in part on a clustering of the extracted plurality of radiomic features.

9. The system of claim 1, wherein the cardiac injury is a myocardial infarction, and wherein the identification of the myocardial infarction is further based at least in part on matching the radiomic features to a gradient of the patient cluster.

10. The system of claim 1, wherein, when executed, the at least one application further causes the at least one computing device to at least determine a global-to-local association and estimate local cardiac damage based at least in part on the global-to-local association.

11. The system of claim 1, wherein, when executed, the at least one application further causes the at least one computing device to at least estimate global cardiac damage based at least in part on the extracted plurality of radiomic features.

12. A method, comprising:
extracting, via at least one computing device, a plurality of radiomic features from an ultrasound scan associated with a person;
identifying, via the at least one computing device, one or more myocardial textures by applying the extracted plurality of radiomic features to at least one phenotyping model;
comparing, via the at least one computing device, the one or more myocardial textures to at least one phenotype cluster for at least one known condition; and
determining, via the at least one computing device, a cardiac injury associated with the person based at least in part on the one or more myocardial textures being matched with one or more of the at least one phenotype cluster, and the extracted plurality of radiomic features.

13. The method of claim 12, wherein the cardiac injury is a myocardial infarction, and further comprising quantifying, via the at least one computing device, a size of an infarct associated with the myocardial infarction based at least in part on the one or more myocardial textures.

14. The method of claim 12, wherein medical images used to determine the cardiac injury consist of one or more ultrasound scans.

15. The method of claim 12, wherein the cardiac injury is a myocardial infarction, and further comprising locating, via the at least one computing device, an infarct associated with the myocardial infarction based at least in part on the one or more myocardial textures.

16. The method of claim 15, further comprising creating, via the at least one computing device, a parametric map of an infarcted myocardium.

17. The method of claim 16, further comprising creating, via the at least one computing device, the parametric map based on a paired cardiac magnetic resonance (CMR) assessment.

18. The method of claim 12, wherein the cardiac injury is a myocardial infarction, and wherein the determination of the myocardial infarction is further based at least in part on matching the radiomic features to a gradient of the at least one phenotype cluster.

19. The method of claim 12, further comprising:
selecting, via the at least one computing device, a portion of the plurality of radiomics features;
selecting, via the at least one computing device, the at least one phenotyping model based at least in part on the portion of the plurality of radiomics features; and
determining the cardiac injury based at least in part on the portion of the plurality of radiomics features and the at least one phenotyping model.

20. The method of claim 12, wherein the ultrasound scan comprises an apical view ultrasound scan and the plurality of features comprise static features and dynamic features.
